(19) United States
(12) Patent Application Publication
(10) Pub. No.: US 2004/0089306 A1
(43) Pub. Date: May 13, 2004

(54) Devices and methods for removing bronchial isolation devices implanted in the lung

(76) Inventors: Ronald Hundertmark, San Mateo, CA (US); Antony Fields, San Francisco, CA (US); Michael Hendricksen, Redwood City, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/448,019

(22) Filed: May 28, 2003

Related U.S. Application Data
(63) Continuation-in-part of application No. 10/270,792, filed on Oct. 10, 2002.
(60) Provisional application No. 60/384,247, filed on May 28, 2002.

(51) Int. Cl. A61M 16/00

(52) U.S. Cl. 128/207.14

(57) ABSTRACT

Disclosed are methods and devices for removing bronchial isolation devices from a lung. According to one method, a flow control device is removed from a bronchial passageway of a patient. A removal device having an elongate shaft and a distal engaging element is provided. The removal device is inserted through the bronchial isolation device such that the distal engaging element is positioned within or distally of the bronchial isolation device in the bronchial passageway. The engaging element is then transitioned to have a radial size that is larger than the radial size of at least a portion of the bronchial isolation device. The bronchial isolation device is engaged with the distal engaging element to urge the bronchial isolation device in the proximal direction, thereby removing the bronchial isolation device out of the bronchial passageway. The bronchial isolation device can also be equipped with a handle that will collapse the bronchial isolation device when force is applied to the handle.

### REFERENCE TO PRIORITY DOCUMENT

This application claims priority of co-pending U.S. Provisional Patent Application Serial No. 60/384,247 entitled “Implantable Bronchial Isolation Devices and Lung Treatment Methods”, filed May 28, 2002. Priority of the aforementioned filing date is hereby claimed, and the disclosure of the Provisional Patent Application is hereby incorporated by reference in its entirety.

This application is a continuation-in-part of U.S. patent application Ser. No. 10/270,792, filed Oct. 10, 2002 and entitled “Bronchial Flow Control Devices and Methods of Use”, which is hereby incorporated by reference in its entirety.

### BACKGROUND OF THE INVENTION

1. Field of the Invention

This invention relates generally to methods and devices for use in performing pulmonary procedures and, more particularly, to procedures for treating lung diseases.

2. Description of the Related Art

Certain pulmonary diseases, such as emphysema, reduce the ability of one or both lungs to fully expel air during the exhalation phase of the breathing cycle. Such diseases are accompanied by chronic or recurrent obstruction to air flow within the lung. One of the effects of such diseases is that the diseased lung tissue is less elastic than healthy lung tissue, which is one factor that prevents full exhalation of air. During breathing, the diseased portion of the lung does not fully recoil due to the diseased (e.g., emphysematic) lung tissue being less elastic than healthy tissue. Consequently, the diseased lung tissue exerts a relatively low driving force, which results in the diseased lung expelling less air volume than a healthy lung.

The problem is further compounded by the diseased, less elastic tissue that surrounds the very narrow airways that lead to the alveoli, which are the air sacs where oxygen-carbon dioxide exchange occurs. The diseased tissue has less tone than healthy tissue and is typically unable to maintain the narrow airways open until the end of the exhalation cycle. This traps air in the lungs and exacerbates the already-inefficient breathing cycle. The trapped air causes the tissue to become hyper-expanded and no longer able to effect efficient oxygen-carbon dioxide exchange.

In addition, hyper-expanded, diseased lung tissue occupies more of the pleural space than healthy lung tissue. In most cases, a portion of the lung is diseased while the remaining part is relatively healthy and, therefore, still able to efficiently carry out oxygen exchange. By taking up more of the pleural space, the hyper-expanded lung tissue reduces the amount of space available to accommodate the healthy, functioning lung tissue. As a result, the hyper-expanded lung tissue causes inefficient breathing due to its own reduced functionality and because it adversely affects the functionality of adjacent healthy tissue.

Lung reduction surgery is a conventional method of treating emphysema. However, such a conventional surgical approach is relatively traumatic and invasive, and, like most surgical procedures, is not a viable option for all patients.

Some recently proposed treatments for emphysema or other lung ailments include the use of devices that isolate a diseased region of the lung in order to modify the air flow to the targeted lung region or to achieve volume reduction or collapse of the targeted lung region. According to such treatments, bronchial isolation devices are implanted in airways feeding the targeted region of the lung. The isolation devices block or regulate fluid flow to the diseased lung region through one or more bronchial passageways that feed air to the targeted lung region. As shown in FIG. 1, the bronchial isolation of the targeted lung region is accomplished by implanting a bronchial isolation device 10 into a bronchial passageway 15 that feeds air to a targeted lung region 20. The bronchial isolation device 10 regulates airflow through the bronchial passageway 15 in which the bronchial isolation device 10 is implanted. The bronchial isolation devices can be, for example, one-way valves that allow flow in the exhalation direction only, occluders or plugs that prevent flow in either direction, or two-way valves that control flow in both directions.

The bronchial isolation device can be implanted in a target bronchial passageway using a delivery catheter that is guided with a guidewire that is placed through the trachea (via the mouth or the nasal cavities) and through the target location in the bronchial passageway. A commonly used technique is to perform what is known as an “exchange technique”, whereby the guidewire is fed through the working channel of a flexible bronchoscope and through the target bronchial passageway. The bronchoscope is then removed from the bronchial tree while leaving the guidewire in place. This is an effective, but somewhat difficult procedure. The guidewire is typically quite long so that it can reach into the bronchial tree, which makes removal of the bronchoscope while keeping the guidewire in place quite difficult. The difficulty arises in that the guidewire can catch onto the inside of the working channel while the bronchoscope is being removed so that the bronchoscope ends up dislodging the guidewire tip from the target bronchial lumen or pulling the guidewire out of the bronchial tree. In view of this difficulty, it would be advantageous to develop an improved method and device for performing the guidewire exchange technique.

In certain circumstances, it is desirable to remove a previously-implanted bronchial isolation device. For example, it may be desirable to remove an implanted device immediately following an implantation procedure, such as where the device has been placed incorrectly or where there is some other problem with the device. It may also be desirable to remove an implanted device as part of a normal therapeutic procedure. Many conventional bronchial isolation devices are not designed for easy removal and, as a consequence, removing such implanted devices can be difficult and costly. Thus, there is a need for improved methods and devices for removing a bronchial isolation device that has been implanted in a bronchial passageway.

As discussed above, one of the major problems experienced by patients who have emphysema is difficulty in fully expelling air from the lungs during exhalation. This is generally due to at least two factors, the loss of elasticity in the lung parenchyma, and the loss of radial tethering on the airways leading to distal airway collapsed during exhalation. Both of these factors make it very difficult for an emphysematic patient to fully exhale from the diseased portions of their lungs. One conventional way of improving this condition is to add additional collateral air channels from the diseased distal lung parenchyma into the proximal airways. The collateral air channels provide alternate routes for air to exit the diseased portion of the lung.

The collateral channels can be formed by cutting or puncturing a channel through the bronchial wall and into the lung tissue or parenchyma. The collateral channels through the bronchial wall are sometimes held open with structures such as stents, grommets or the like. As mentioned, the addition of the collateral channels allows trapped gas in the distal lung tissue to be vented much more easily upon exhalation. However, these collateral channels might undesirably also allow more air to flow into the diseased tissue during inhalation, and may increase hyperinflation, and especially dynamic hyperinflation (hyperinflation during exertion). Thus, there is a need for devices and methods for regulating air flow through collateral air channels into the diseased lung region.

### SUMMARY

Disclosed is a method of removing a flow control device implanted in a bronchial passageway of a patient. The method comprises providing a removal device having an elongate shaft and a distal engaging element; inserting the removal device through the bronchial isolation device such that the distal engaging element is positioned within or distally of the bronchial isolation device in the bronchial passageway; transitioning the engaging element of the removal device to have a radial size that is larger than the radial size of at least a portion of the bronchial isolation device; and engaging the bronchial isolation device with the distal engaging element to urge the bronchial isolation device in the proximal direction, thereby removing the bronchial isolation device out of the bronchial passageway.

Also disclosed is a removable flow control device for implanting in a bronchial passageway. The devices comprises a valve member that regulates fluid flow through the flow control device; a seal member that at least partially surrounds the valve member, wherein the seal member seals with the interior wall of the bronchial passageway when the flow control device is implanted in the bronchial passageway; a retainer member secured to the seal member, wherein the retainer member exerts a radial force against the interior wall of the bronchial passageway when the flow control device is implanted in the bronchial passageway and retains the flow control device in a fixed location in the bronchial passageway; and a removal handle attached to the retainer member, the handle being configured to collapse the retainer member upon application of a force to the handle member.

Other features and advantages of the present invention should be apparent from the following description of various embodiments, which illustrate, by way of example, the principles of the invention.

### BRIEF DESCRIPTION OF THE DRAWINGS

FIG. 1 shows an anterior view of a pair of human lungs and a bronchial tree with a bronchial isolation device implanted in a bronchial passageway to bronchially isolate a region of the lung.

FIG. 2 illustrates an anterior view of a pair of human lungs and a bronchial tree.

FIG. 3 illustrates a lateral view of the right lung.

FIG. 4 illustrates a lateral view of the left lung.

FIG. 5 illustrates an anterior view of the trachea and a portion of the bronchial tree.

FIG. 6 shows a guidewire delivery system that can be used to deliver a guidewire to a location in a bronchial passageway, wherein the guidewire is located externally to a delivery device.

FIG. 7 shows a perspective view of a bronchoscope.

FIG. 8 shows an enlarged view of a distal region of a bronchoscope.

FIG. 9 shows a first embodiment of a guidewire grasping tool.

FIG. 10A shows an enlarged view a distal region of the grasping tool of FIG. 9 protruding through the distal end of a working channel of a bronchoscope.

FIG. 10B shows an enlarged view a distal region of another embodiment of the grasping tool of FIG. 9 protruding through the distal end of a working channel of a bronchoscope.

FIG. 11A shows an enlarged view a distal region of another embodiment of a grasping tool protruding through the distal end of a working channel of a bronchoscope.

FIG. 11B shows an enlarged view of the distal region of the grasping tool of FIG. 11 protruding through the distal end the bronchoscope and grasping a guidewire.

FIG. 12A shows an enlarged view of the distal region of the grasping tool of FIG. 11 in an open state.

FIG. 12B shows an enlarged view of the distal region of the grasping tool of FIG. 11 in a closed state.

FIG. 13 shows another embodiment of a grasping tool.

FIG. 14 shows an enlarged view of the distal region of the grasping tool of FIG. 13 protruding through the distal end the bronchoscope and grasping a guidewire.

FIG. 15 shows an enlarged view of the distal region of the grasping tool of FIG. 13 protruding through the distal end the bronchoscope and grasping the guidewire.

FIG. 16 shows an enlarged view of the distal region of the grasping tool of FIG. 13 protruding through the distal end the bronchoscope and releasing the guidewire.

FIG. 17A shows a perspective view of another embodiment of a grasping tool comprising a clip located on a distal region of a bronchoscope.

FIG. 17B shows a perspective view of another embodiment of the clip grasping tool located on a distal region of a bronchoscope.

FIG. 17C shows a perspective view of yet another embodiment of the clip grasping tool located on a distal region of a bronchoscope.

FIG. 18 shows a side view of the grasping tool and bronchoscope of FIG. 17A.

FIG. 19 shows a cross-sectional view of the grasping tool of FIG. 17A cut along line A-A of FIG. 18.

FIG. 20 shows a perspective view of yet another embodiment of the clip grasping tool located on a distal region of a bronchoscope.

FIG. 21 shows a side view of the grasping tool and bronchoscope of FIG. 20.

FIG. 22 shows a cross-sectional view of the grasping tool of FIG. 20 cut along line A-A of FIG. 21.

FIG. 23 shows a perspective view of yet another embodiment of a grasping tool comprising plural clips located on a distal region of a bronchoscope.

FIG. 24 shows a perspective view of an exemplary embodiment of a bronchial isolation device.

FIG. 25 shows a cross-sectional side view of the bronchial isolation device of FIG. 24.

FIG. 26 shows a side view of the bronchial isolation device of FIG. 24.

FIG. 27 shows a bronchial isolation device mounted in a bronchial passageway and crossed by a removal device.

FIG. 28 shows a perspective view of a bronchial isolation device having a removal handle.

FIG. 29 shows a bronchial isolation device having multiple removal handles mounted in a bronchial passageway.

FIG. 30 shows a side view of a bronchial isolation device having another embodiment of a removal handle.

FIG. 31 shows a side view of a bronchial isolation device having another embodiment of a removal handle.

FIG. 32 shows an anchor member having a plurality of eyelets for attaching to a removal handle.

FIG. 33 shows another embodiment of a bronchial isolation device mounted in a bronchial passageway.

FIG. 34 shows a shape-changing removal ring member that is attached to the proximal end of the valve protector wherein the removal ring is in a widened state.

FIG. 35 shows the shape-changing removal ring member wherein the removal ring is in a constricted state.

FIG. 36 shows the shape-changing removal ring member in a constricted state and part of a bronchial isolation device.

FIG. 37 shows a cutaway view of the human right lung with a number of channels cut through the walls of bronchial passageways such that the channels fluidly connect the upper lobar bronchus and segmental bronchus directly to the lung parenchyma.

FIG. 38 shows a cutaway view of the human right lung with flow control devices mounted in channels cut through the walls of bronchial passageways.

FIG. 39 shows a cross-sectional side view of a flow control device for mounting in a channel cut through the wall of a bronchial passageway.

FIG. 40A shows a cross-sectional side view of the flow control device of FIG. 39 mounted in a channel cut through the wall of a bronchial passageway.

FIG. 40B shows a cross-sectional side view of another embodiment of a flow control device mounted in a channel cut through the wall of a bronchial passageway.

FIG. 41 shows another embodiment of a flow control device for mounting in a channel cut through the wall of a bronchial passageway.

FIG. 42 shows a cross-sectional side view of the flow control device of FIG. 41 mounted in a channel cut through the wall of a bronchial passageway.

FIG. 43A shows a planar view of an embodiment of the retainer member of the flow control device of FIG. 42 in a pre-assembled state.

FIG. 43B shows a planar view of another embodiment of the retainer member of the flow control device of FIG. 42 in a pre-assembled state.

FIG. 44 shows another embodiment of a flow control device for mounting in a channel cut through the wall of a bronchial passageway.

FIG. 45 shows another embodiment of a flow control device for mounting in a channel cut through the wall of a bronchial passageway.

FIG. 46 shows another embodiment of a flow control device for mounting in a channel cut through the wall of a bronchial passageway.

FIG. 47 shows a perspective view of a seal and retainer member of the flow control device shown in FIG. 45.

FIG. 48 shows another embodiment of a flow control device for mounting in a channel cut through the wall of a bronchial passageway.

FIG. 49 shows a perspective view of a seal and retainer member of the flow control device shown in FIG. 48.

FIG. 50 shows another embodiment of a flow control device for mounting in a channel cut through the wall of a bronchial passageway.

FIG. 51 shows a cutaway, perspective view looking into a bronchial passageway in which the flow control device of FIG. 50 is mounted.

FIG. 52 shows a cutaway, perspective view looking into a bronchial passageway in which another embodiment of the flow control device of FIG. 50 is mounted.

FIG. 53 shows a perspective view of a diaphragm of the flow control device of FIG. 52.

FIG. 54 shows a cross-sectional view of the diaphragm of FIG. 53.

FIG. 55 shows a cutaway view of the human right lung with flow control devices mounted in channels cut through the walls of bronchial passageways and bronchial isolation devices mounted within the bronchial passageways.

FIG. 56 shows a cutaway view of the human right lung with flow control devices mounted in channels cut through the walls of bronchial passageways and bronchial isolation devices mounted within the bronchial passageways.

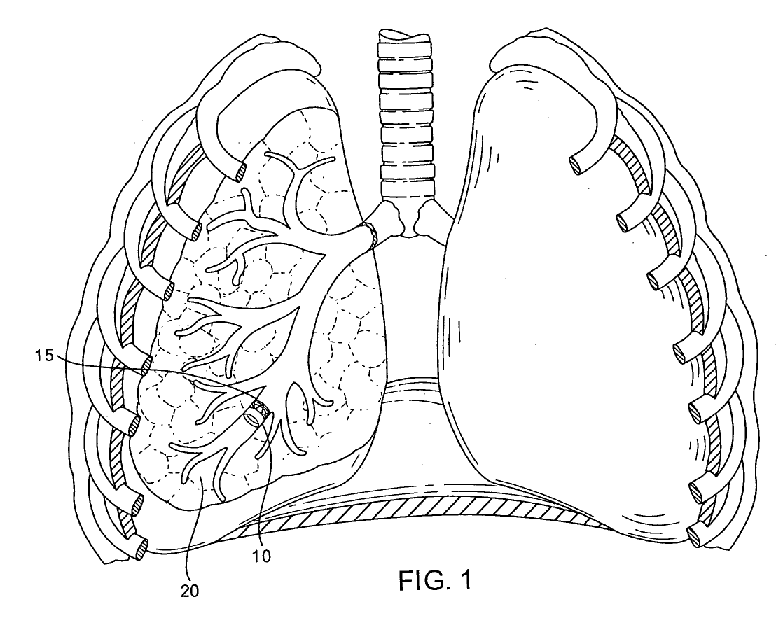

### DETAILED DESCRIPTION

Unless defined otherwise, all technical and scientific terms used herein have the same meaning as is commonly understood by one of skill in the art to which the invention(s) belong. Disclosed are various devices and method for treating bronchopulmonary diseases.

Exemplary Lung Regions

Throughout this disclosure, reference is made to the term “lung region”. As used herein, the term “lung region” refers to a defined division or portion of a lung. For purposes of example, lung regions are described herein with reference to human lungs, wherein some exemplary lung regions include lung lobes and lung segments. Thus, the term “lung region” as used herein can refer, for example, to a lung lobe or a lung segment. Such nomenclature conform to nomenclature for portions of the lungs that are known to those skilled in the art. However, it should be appreciated that the term “lung region” does not necessarily refer to a lung lobe or a lung segment, but can refer to some other defined division or portion of a human or non-human lung.

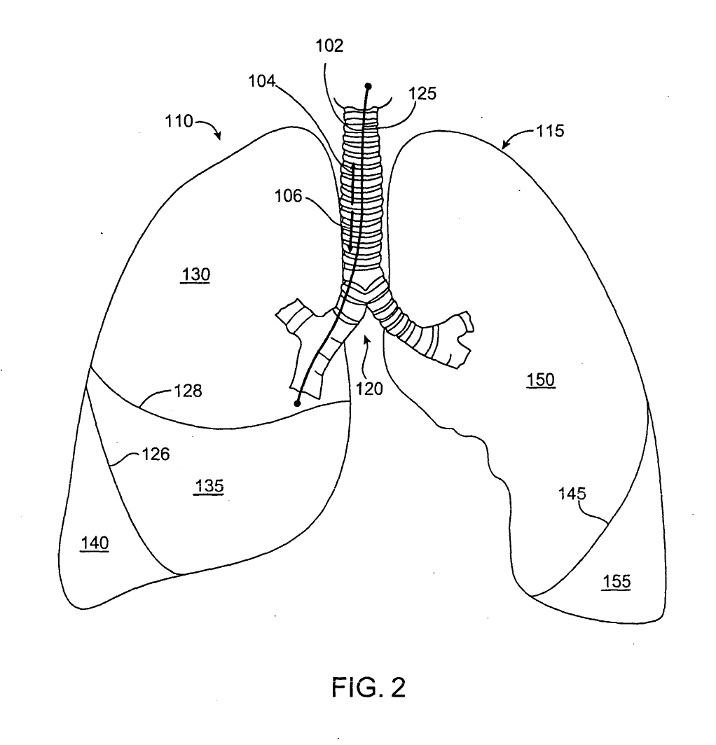

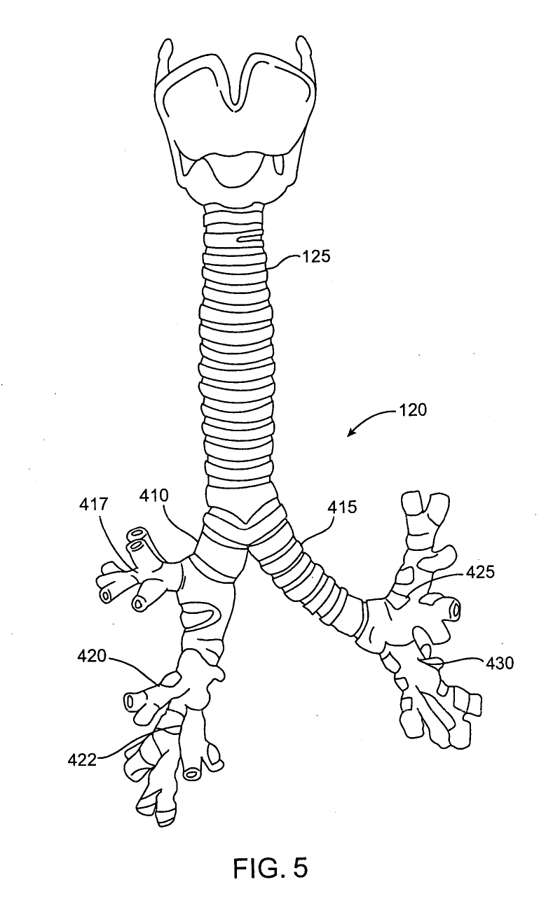

FIG. 2 shows an anterior view of a pair of human lungs 110, 115 and a bronchial tree 120 that provides a fluid pathway into and out of the lungs 110, 115 from a trachea 125, as will be known to those skilled in the art. As used herein, the term “fluid” can refer to a gas, a liquid, or a combination of gas(es) and liquid(s). For clarity of illustration, FIG. 2 shows only a portion of the bronchial tree 120, which is described in more detail below with reference to FIG. 5.

Throughout this description, certain terms are used that refer to relative directions or locations along a path defined from an entryway into the patient's body (e.g., the mouth or nose) to the patient's lungs. The path of airflow into the lungs generally begins at the patient's mouth or nose, travels through the trachea into one or more bronchial passageways, and terminates at some point in the patient's lungs. For example, FIG. 2 shows a path 102 that travels through the trachea 125 and through a bronchial passageway into a location in the right lung 110. The term “proximal direction” refers to the direction along such a path 102 that points toward the patient's mouth or nose and away from the patient's lungs. In other words, the proximal direction is generally the same as the expiration direction when the patient breathes. The arrow 104 in FIG. 2 points in the proximal or expiratory direction. The term “distal direction” refers to the direction along such a path 102 that points toward the patient's lung and away from the mouth or nose. The distal direction is generally the same as the inhalation or inspiratory direction when the patient breathes. The arrow 106 in FIG. 2 points in the distal or inhalation direction.

The lungs include a right lung 110 and a left lung 115. The right lung 110 includes lung regions comprised of three lobes, including a right upper lobe 130, a right middle lobe 135, and a right lower lobe 140. The lobes 130, 135, 140 are separated by two interlobar fissures, including a right oblique fissure 126 and a right transverse fissure 128. The right oblique fissure 126 separates the right lower lobe 140 from the right upper lobe 130 and from the right middle lobe 135. The right transverse fissure 128 separates the right upper lobe 130 from the right middle lobe 135.

As shown in FIG. 2, the left lung 115 includes lung regions comprised of two lobes, including the left upper lobe 150 and the left lower lobe 155. An interlobar fissure comprised of a left oblique fissure 145 of the left lung 115 separates the left upper lobe 150 from the left lower lobe 155. The lobes 130, 135, 140, 150, 155 are directly supplied air via respective lobar bronchi, as described in detail below.

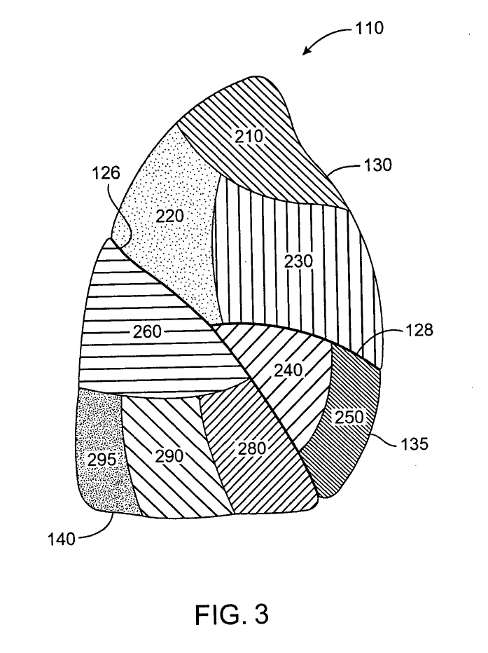

FIG. 3 is a lateral view of the right lung 110. The right lung 110 is subdivided into lung regions comprised of a plurality of bronchopulmonary segments. Each bronchopulmonary segment is directly supplied air by a corresponding segmental tertiary bronchus, as described below. The bronchopulmonary segments of the right lung 110 include a right apical segment 210, a right posterior segment 220, and a right anterior segment 230, all of which are disposed in the right upper lobe 130. The right lung bronchopulmonary segments further include a right lateral segment 240 and a right medial segment 250, which are disposed in the right middle lobe 135. The right lower lobe 140 includes bronchopulmonary segments comprised of a right superior segment 260, a right medial basal segment (which cannot be seen from the lateral view and is not shown in FIG. 3), a right anterior basal segment 280, a right lateral basal segment 290, and a right posterior basal segment 295.

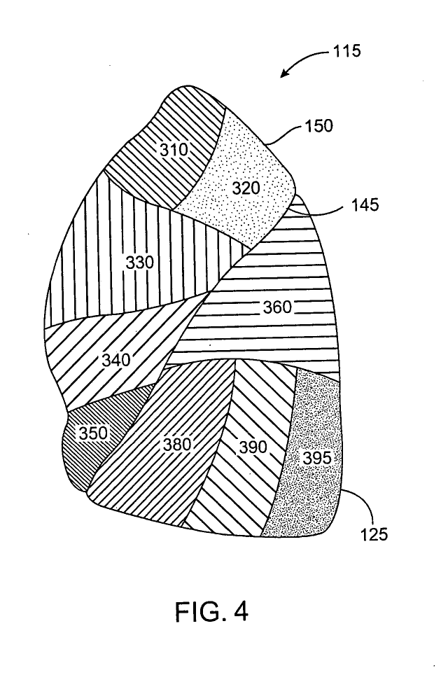

FIG. 4 shows a lateral view of the left lung 115, which is subdivided into lung regions comprised of a plurality of bronchopulmonary segments. The bronchopulmonary segments include a left apical segment 310, a left posterior segment 320, a left anterior segment 330, a left superior segment 340, and a left inferior segment 350, which are disposed in the left lung upper lobe 150. The lower lobe 155 of the left lung 115 includes bronchopulmonary segments comprised of a left superior segment 360, a left medial basal segment (which cannot be seen from the lateral view and is not shown in FIG. 4), a left anterior basal segment 380, a left lateral basal segment 390, and a left posterior basal segment 395.

FIG. 5 shows an anterior view of the trachea 125 and a portion of the bronchial tree 120, which includes a network of bronchial passageways, as described below. In the context of describing the lung, the terms “pathway” and “lumen” are used interchangeably herein. The trachea 125 divides at a lower end into two bronchial passageways comprised of primary bronchi, including a right primary bronchus 410 that provides direct air flow to the right lung 110, and a left primary bronchus 415 that provides direct air flow to the left lung 115. Each primary bronchus 410, 415 divides into a next generation of bronchial passageways comprised of a plurality of lobar bronchi. The right primary bronchus 410 divides into a right upper lobar bronchus 417, a right middle lobar bronchus 420, and a right lower lobar bronchus 422. The left primary bronchus 415 divides into a left upper lobar bronchus 425 and a left lower lobar bronchus 430. Each lobar bronchus, 417, 420, 422, 425, 430 directly feeds fluid to a respective lung lobe, as indicated by the respective names of the lobar bronchi. The lobar bronchi each divide into yet another generation of bronchial passageways comprised of segmental bronchi, which provide air flow to the bronchopulmonary segments discussed above.

As is known to those skilled in the art, a bronchial passageway defines an internal lumen through which fluid can flow to and from a lung or lung region. The diameter of the internal lumen for a specific bronchial passageway can vary based on the bronchial passageway's location in the bronchial tree (such as whether the bronchial passageway is a lobar bronchus or a segmental bronchus) and can also vary from patient to patient. However, the internal diameter of a bronchial passageway is generally in the range of 3 millimeters (mm) to 10 mm, although the internal diameter of a bronchial passageway can be outside of this range. For example, a bronchial passageway can have an internal diameter of well below 1 mm at locations deep within the lung.

Guidewire Delivery System

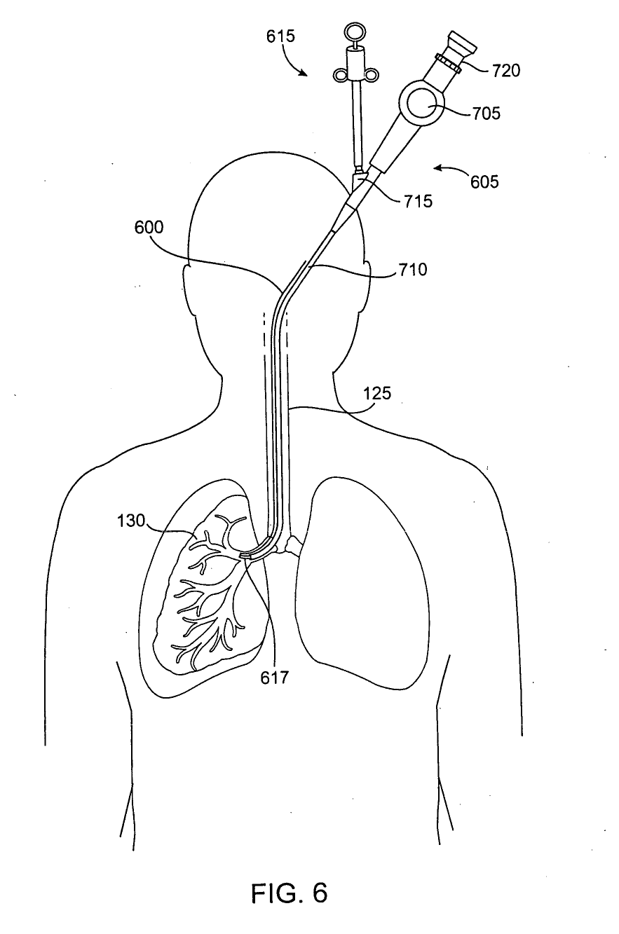

FIG. 6 shows a delivery system that can be used to deliver a guidewire 600 to a location in a bronchial passageway according to an exchange technique. A distal end of the guidewire 600 is fixed to the outside of a guidewire delivery device, such as a bronchoscope 605, that has been passed through a patient's trachea 125 and into the right lung 130. A guidewire grasping tool 615 is coupled to the bronchoscope such that a grasping member 617 on a distal end of the grasping tool 615 grasps a distal end of the guidewire to secure the guidewire to the bronchoscope, as described in more detail below.

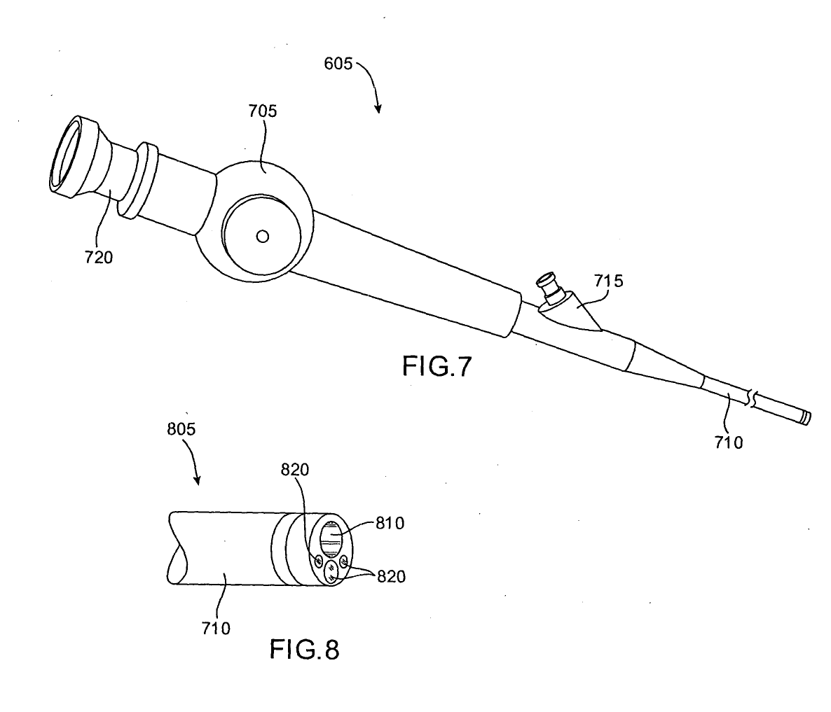

FIG. 7 shows an enlarged view of the bronchoscope 605, which in an exemplary embodiment has a steering mechanism 705, a delivery shaft 710, a working channel entry port 715, and a visualization eyepiece 720. In addition, the bronchoscope can also include a fiber optic bundle mounted inside the length of the bronchoscope for transferring an image from the distal end to the eyepiece 720. In one embodiment, the bronchoscope also includes a camera or charge-coupled device (CCD) for generating an image of the bronchial tree. As described below, the guidewire grasping tool 615 can be coupled to the bronchoscope via the entry port 715. FIG. 8 shows an enlarged view of the distal portion 805 of the bronchoscope 605. A working channel 810 extends through the shaft 710 and communicates with the entry port 715 (shown in FIG. 7) at the proximal end of the bronchoscope 605. The bronchoscope 605 can also include various other channels 820.

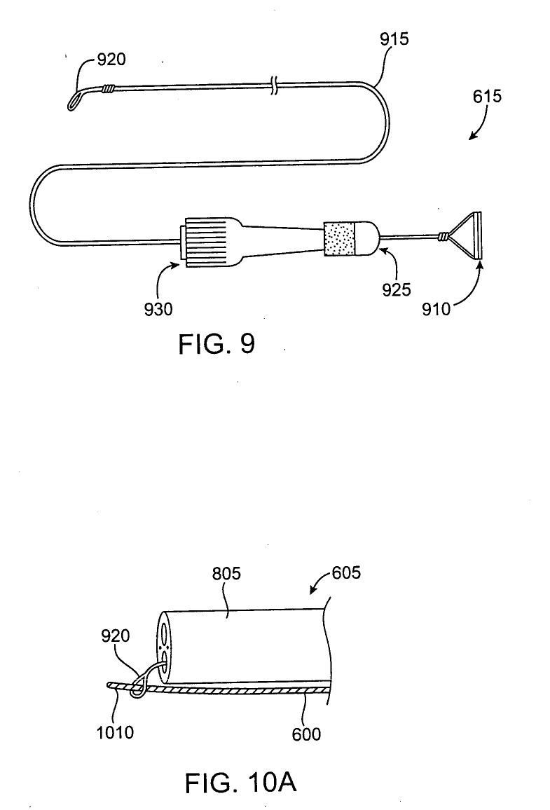

FIG. 9 shows a first embodiment of the guidewire grasping tool 615, which includes a proximal actuator handle 910 that is attached to an elongate grasping wire 915 that is sized to be positioned within the working channel 810 of the bronchoscope 605. That is, the elongate grasping wire 915 has a diameter that is less than the diameter of the working channel 810. The elongate grasping wire 915 is sufficiently long such that the distal end of the elongate grasping wire 915 can protrude outwardly from the distal end of the working channel 810 when the guidewire grasping tool 615 is coupled to the bronchoscope 605, as described below.

With reference to FIG. 9, a grasping member comprised of a grasping loop 920 is located at the distal end of the elongate grasping wire 915. The grasping loop 920 is of sufficient size to receive the guidewire 600 therethrough. The grasping tool 920 also includes a torquer 925 that is attached to the elongate grasping wire 915. The torquer 925 can be rotated to apply torque to the grasping wire 915 and cause the grasping wire 915 (and the grasping loop 920) to rotate. A coupler 930 is located near the actuator handle 910 for removably coupling the guidewire grasping tool 615 to the bronchoscope. The coupler can comprise, for example, a connection component (such as a Luer fitting) that couples to a corresponding connection component on the working channel entry port 715 (shown in FIG. 7).

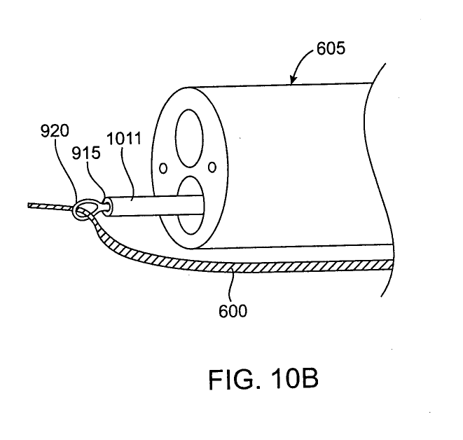

In use, the distal end of the grasping wire 915 is inserted into the working channel 810 of the bronchoscope 605, such as by inserting the grasping loop 920 through the working channel entry port 715. The grasping wire 915 is then fed through the working channel 810 so that the grasping loop 920 protrudes through the distal end of the working channel 810, as shown in FIG. 10A. The actuation handle 910 is then coupled to the bronchoscope by attaching the coupler 930 on the grasping tool to the corresponding coupler on the working channel entry port 715. As mentioned, the grasping wire 915 is sufficiently long so that the grasping loop 920 can protrude outwardly from the distal end of the working channel 810 when the coupler 930 is coupled to the working channel entry port 715.

When the guidewire grasping tool 615 is coupled to the bronchoscope 605, the distal end 1010 of the guidewire 600 is inserted through the grasping loop 920 so that the length of the guidewire 600 is positioned adjacent the length of the elongate shaft 710 of the bronchoscope 605, as shown generally in FIG. 6 and in a close-up of the distal region in FIG. 10A. In one embodiment, the actuator handle 910 can be pulled in a proximal direction, which moves the grasping loop 920 proximally into the working channel 810. As the grasping loop 920 moves into the working channel, it tightens around the guidewire 600 and holds it in place against the distal end of the bronchoscope. In another embodiment, shown in FIG. 10B, the grasping wire 915 is slidably mounted within an elongate tube 1011. The grasping wire 915 can be pulled distally relative to the elongate tube 1011, which draws the grasping loop 920 into the elongate tube 1011 and tightens the grasping loop 920 around the guidewire 600.

With the distal ends of the guidewire 600 and the bronchoscope 605 secured to one another as such, the bronchoscope 605 and the attached guidewire 600 can then be fed through the patient's trachea until the distal end of the bronchoscope 605 locates at a desired location within the bronchial tree, as shown in FIG. 6. In this manner, the distal end 1010 of the guidewire 600 is also located at the desired location within the bronchial tree. Once the guidewire is in place, the guidewire grasping tool 615 can be released from the guidewire 600 by loosening the loop's grip on the guidewire. This can be accomplished by removing tension from the grasping wire 915 to loosen the grasping loop's hold on the distal end of the guidewire 600. The bronchoscope 605 can then be withdrawn, leaving the guidewire 600 in place.

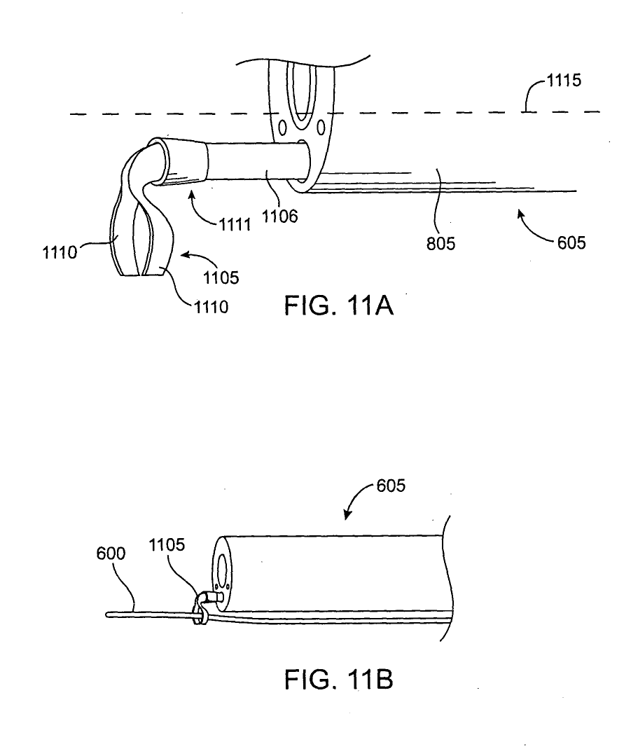

FIGS. 11A and 11B shows another embodiment of the guidewire grasping tool 615. In this embodiment, the grasping member comprises forceps 1105 that are located at the distal end of an elongate grasping sleeve 1106 (rather than at the end of a grasping wire as in the previous embodiment). The forceps 1105 include two or more fingers 1110 that are slidably mounted within a collar 1111 on the distal end of the sleeve 1106. As shown in FIG. 11B, the guidewire 600 can be grasped by the forceps 1105 and secured to the distal end of the bronchoscope 605. FIGS. 11A and 11B show the distal end of the sleeve 1106 being generally straight and generally parallel with the length of the bronchoscope 605. It should be appreciated that the distal end of the sleeve 1106 can also have a curved configuration.

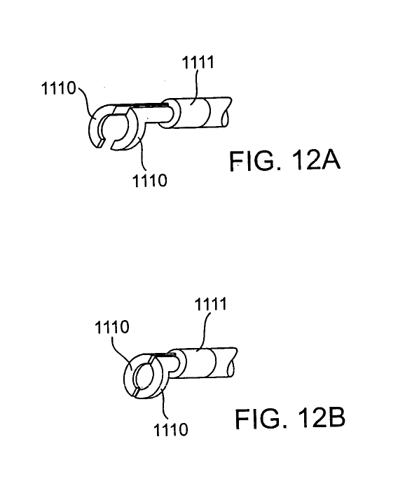

FIGS. 12A and 12B show how the fingers 1110 can be opened and closed relative to one another. The fingers 1110 are biased to move away from one another when unopposed. As shown in FIG. 12A, the fingers 1110 can be moved in the distal direction relative to the collar 1111 by pushing on the handle 910 or otherwise actuating the handle 910 in some manner. In this position, the collar 1111 does not constrict the fingers together so that the bias causes the fingers 1110 to separate from one another. As shown in FIG. 12B, the fingers 1110 can be pulled in the proximal direction relative to the collar 1111 by pulling on the actuator handle 910, which is coupled to the fingers 1110 via an elongate connector slidably positioned in the sleeve 1106. When the fingers are moved in the proximal direction, they move deeper into the collar 1111, which constricts the fingers 1110 toward one another to decrease the size of the space between the fingers. The default state of the fingers 1110 is to move away from one another. Thus, when the fingers are moved in the distal direction (by manipulating the actuator handle 910), the fingers 1110 slide out of the tapered collar 111 and separate from one another, as the tapered collar 1111 no longer constricts the fingers.

In the embodiment shown in FIGS. 11-12, the fingers 1110 extend along a direction that is transverse to the longitudinal axis 1115 of the bronchoscope shaft 710. In this manner, the fingers 1110 can hold the guidewire 600 at a location that is adjacent an outer surface of the shaft 710, as shown in FIG. 11B.

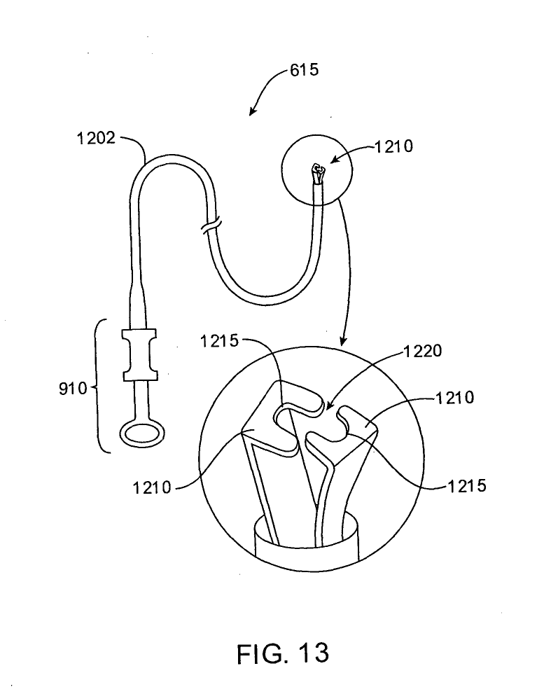

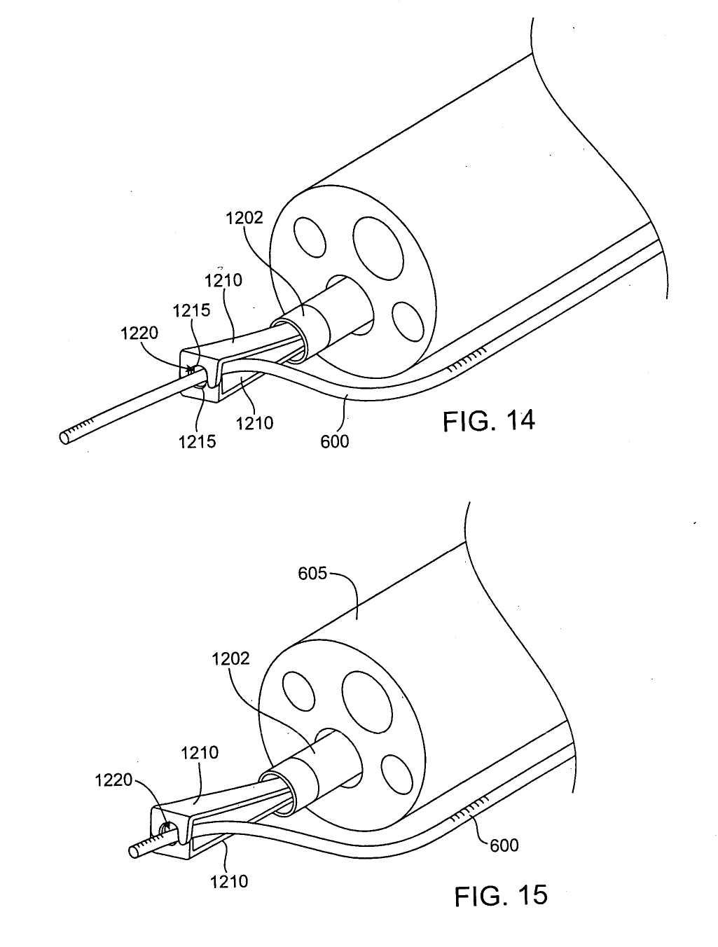

FIG. 13 shows another embodiment of the guidewire grasping tool 615, which includes the actuator handle 910, an elongate sleeve 1202, and a gasping member comprised of a pair of opposed grasping jaws 1210. The grasping jaws 1210 are attached to the actuator handle 910 through the sleeve 1205, as described below. Each grasping jaw 1210 has a notch 1215. In the illustrated embodiment, the notches 1215 are v-shaped or u-shaped, although the notches 1215 can have other shapes configured to receive the guidewire 600. The notches 1215 collectively form an eyelet or opening 1220, the size of which can be varied by moving the jaws 1210 toward or away from one another, such as by manipulating the actuator handle 910. As shown in FIG. 14, the distal end of the guidewire 600 is inserted through the opening 1220 formed by the notches 1215 so that the guidewire 600 is grasped by the jaws 1210. As shown in FIG. 15, the jaws 1210 can be drawn toward one another to decrease the size of the opening 1220 and tightly secure the guidewire 600 between the jaws. Advantageously, the guidewire is held at a point in axial alignment with the bronchoscope to facilitate placement.

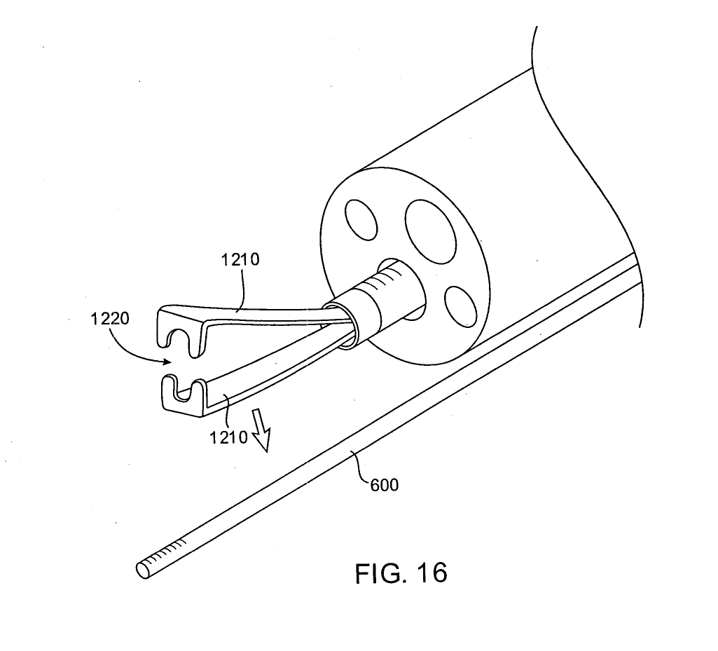

In one embodiment, the grasping jaws 1210 are biased away from one another such that their default state is to move away from one another unless otherwise inhibited. The actuator handle 910 can be pulled in a proximal direction to pull the jaws 1210 deeper into the sleeve 1202 so that the sleeve 1202 effectively compresses the jaws 1210 toward one another. The actuator handle 910 can also be pushed in a distal direction to move the jaws 1210 distally and outwardly of the sleeve 1202. The jaws 1210 are then released from the sleeve 1202 so that the bias causes the jaws to move away from one another and increase the size of the opening 1220, as shown in FIG. 16. The guidewire 600 can be released from the jaws 1210 by increasing the size of the opening 1220 to a sufficient size. Alternately, the jaws 1210 may be actuated in other ways.

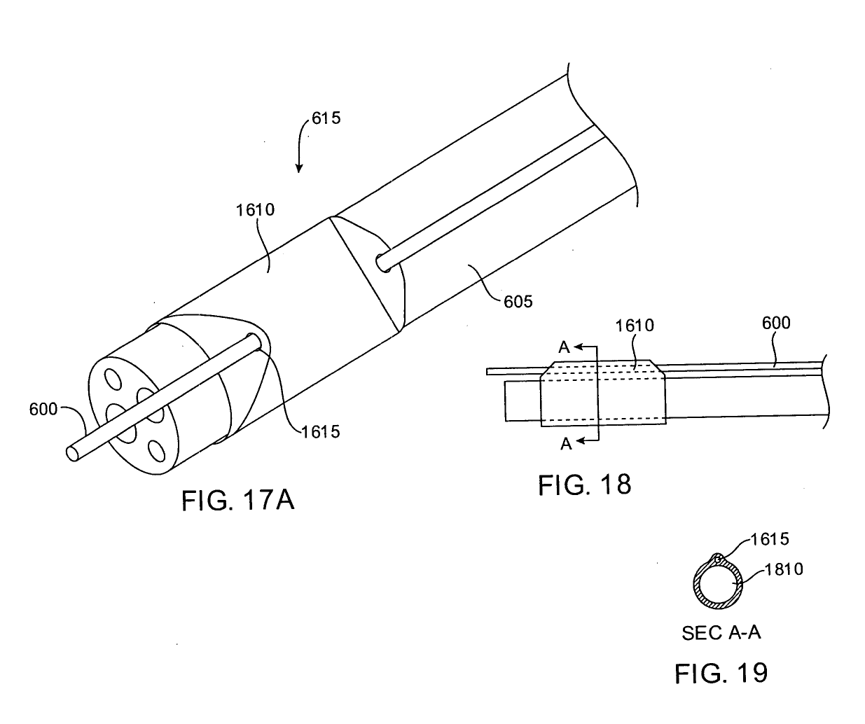

FIGS. 17-23 show another embodiment of the guidewire grasping tool 615, wherein the grasping tool 615 comprises a clip or ring 1610 located on the distal end of a flexible bronchoscope 605 to hold the guidewire 600 along the side of the bronchoscope 605 during placement of the guidewire in the bronchial tree. In the embodiment shown in FIG. 17A, the clip 1610 has a length that provides the clip 1610 with an oblong shape. As shown in FIG. 17B, the length of the clip 1610 can also be shorter so that the clip 1610 has a disc-like appearance. It should be appreciated that the length of the clip 1610 can vary. With reference to FIGS. 17A and 17B, the clip 1610 has small side guidewire lumen 1615 through which the guidewire 600 is passed. The clip 1610 can be made of a flexible and elastic material, such as molded silicone. It should be appreciated that the guidewire lumen 1615 can also be formed by a protrusion that extends outwardly from the side of the bronchoscope to form an eyelet.

FIG. 17C shows another embodiment of the clip 1610, wherein the guidewire lumen 1615 is formed by a series of open-ended channels 1709 in the clip 1610. The channels 1709 are c-shaped, with the open end of each successive channel 1709 facing a different direction. In this manner, the channels 1709 collectively form a passageway in which the guidewire 600 can be mounted. The guidewire 600 does not have to be threaded into the passageway. Rather, the guidewire 600 can be inserted into the open ends of the channels 1709.

As shown in FIG. 19, the clip 1610 includes a hole 1810 for mounting the clip 1610 onto the bronchoscope 615. The hole 1810 is sized to receive the distal region of the bronchoscope 605, such as in a press fit fashion. The clip 1610 can be annular, such as shown in FIG. 19, so as to be slidable over the end of the bronchoscope 615. Alternately, the clip 1610 can be partially annular to allow the clip 1610 to be mounted on the bronchoscope from the side. Alternately, the clip 1610 can be integrally formed with the bronchoscope 605.

The clip 1610 retains the guidewire 600 in place relative to the bronchoscope 605. This prevents the guidewire 600 from moving relative to the bronchoscope 605 while the scope/wire combination is advanced through the bronchial tree to the target location. If sufficient force is applied to the guidewire 600, the guidewire 600 can also be slidably moved through the lumen 1615. Thus, the clip 1610 also allows the guidewire to be released once the scope/wire combination has been advanced to the desired bronchial location. Thus, the guidewire 600 may be left behind in the bronchial passageway when the bronchoscope 605 is withdrawn.

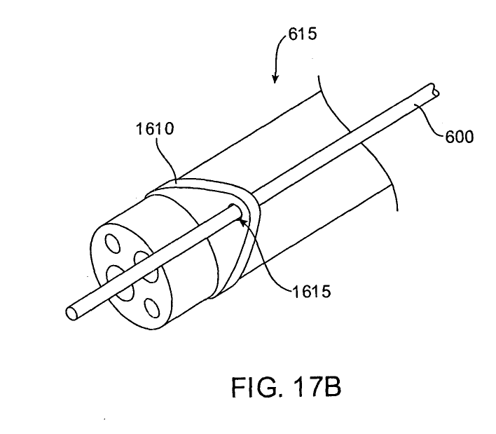

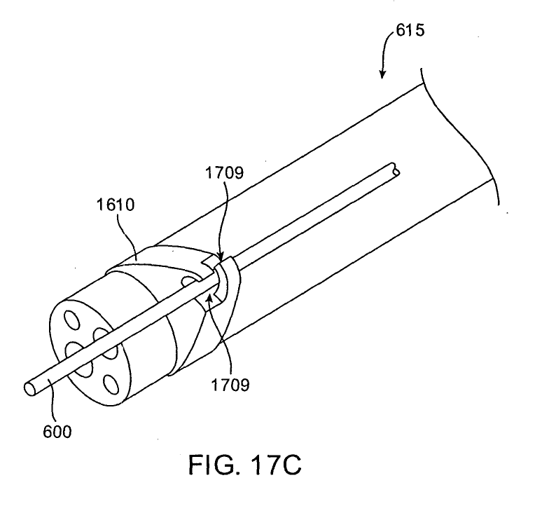

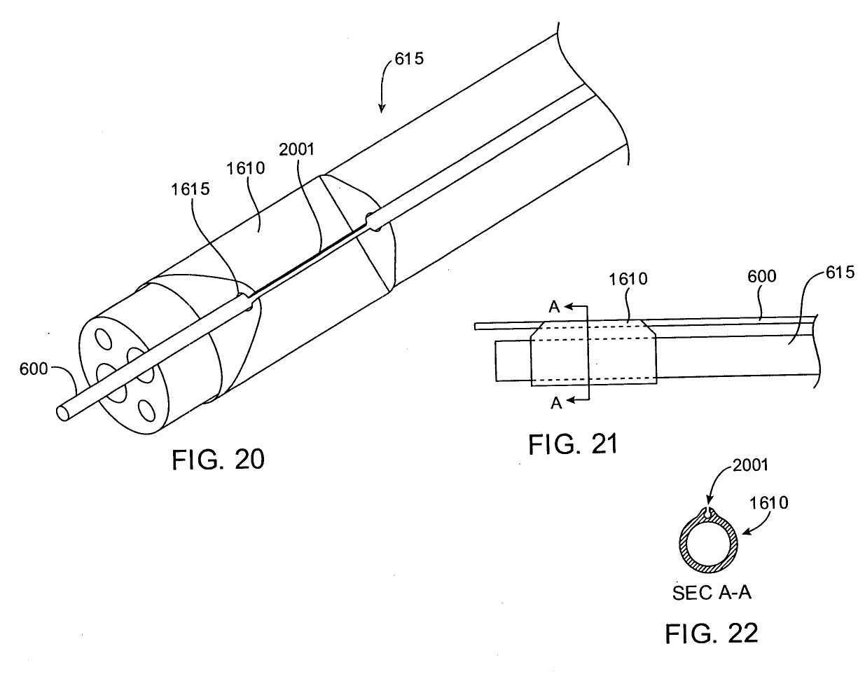

FIGS. 20-22 show another embodiment of the clip 1610 in which an elongate slit 2001 is located in the clip 1610 along the length of the lumen 1615. The slit 2001 communicates with the lumen 1615 to provide an opening through which the guidewire 600 can be inserted into the lumen 1615 or pulled out of the lumen 1615 from the side. This eliminates the need to thread the guidewire lengthwise into the lumen 1615. The clip 1610 shown in FIGS. 20-22 may be made of an elastomer material to provide some flexibility in the region of the slit 2001.

In one embodiment, the guidewire 600 has a radially-enlarged distal region, such as a small protrusion, bump, or the like, to provide the guidewire 600 with a sufficiently large diameter relative to the lumen 1615 such that it cannot inadvertently slide out of the lumen 1615. The enlarged region of the guidewire 600 provides a slight detent with respect to the lumen 600 so that the guidewire 600 can be advanced along with the bronchoscope through the bronchial tree without the guidewire 600 inadvertently slipping out of the lumen 1615. However, when the guidewire 600 is in the desired bronchial position, the bronchoscope 605 can be pulled proximally from the bronchial tree over the guidewire to leave the guidewire in place.

In another embodiment, the diameter of the lumen 1615 is oversized relative to the diameter of the guidewire 600, and the lumen 1615 is lined with a low friction material, such as PTFE, to allow the guidewire 600 to slide smoothly and freely through the lumen 1615. In this embodiment, an operator can manually hold the guidewire 600 to the bronchoscope 605 at the bronchoscope handle. Once the guidewire 600 is in position in the bronchial passageway, the bronchoscope 605 is removed easily and the guidewire left in place. Given that the guidewire 600 is connected to the bronchoscope 605 only at or near the distal end of the bronchoscope, the guidewire 600 may be held manually at the location where the guidewire enters the patient's body (either the nose or the mouth) during withdrawal of the bronchoscope 605. This makes the removal of the bronchoscope much easier after placement, and greatly reduces the chance of dislodging the guidewire.

In an alternative embodiment, the guidewire grasping tool 615 comprises two or more clips 1610 that secure an elongate guidewire tube 2210 to the side of the bronchoscope, as shown in FIG. 23. The guidewire tube 2210 is positioned in the lumens 1615 of the clips 1610. The clips 1610 hold the tube 2210 in a position substantially adjacent the side of the bronchoscope 605 such that the tube 2210 is aligned substantially parallel to the bronchoscope 605. The guidewire 600 is inserted lengthwise through the tube 2210 such that the distal end of the guidewire 600 protrudes distally out of the tube 2210. The tube 2210 can have a slit that provides an opening into the tube 2210 so that the guidewire 600 can be inserted or removed through the side of the tube 2210.

The clips 1610 can be set at a variety of distances from one another. In one embodiment, the most proximal clip on the bronchoscope is spaced a maximum of 60 centimeters from the most distal clip on the bronchoscope. In another embodiment, the most proximal clip is spaced at least 10 cm from the most distal clip. It should be appreciated, however, that the spacing between the most proximal and the most distal clip can vary. The most distal clip can be located a variety of distances from the distal end of the bronchoscope.

The guidewire tube 2210 can be made of or lined with a low friction material, such as PTFE, to allow the guidewire 600 to slide freely through the tube. In one embodiment, the guidewire tube 2210 is flexible enough to allow it to bend freely when the distal tip of the bronchoscope 605 is deflected. In one embodiment, the clips 1610 are manufactured of an elastomer, such as silicone. It should be appreciated that the multiple clips 1610 can also be used without the tube 2210 so that the guidewire 600 is positioned directly in the lumens 1615 of the multiple clips 1610 rather than within the tube 2210. The tube 2210 can also be used with a single clip 1610 rather than with multiple clips.

Exemplary Bronchial Isolation Devices

As discussed above, a target lung region can be bronchially isolated by advancing a bronchial isolation device into the one or more bronchial pathways that feed air to and from the targeted lung region. The bronchial isolation device can be a device that regulates the flow of fluid into or out of a lung region through a bronchial passageway.

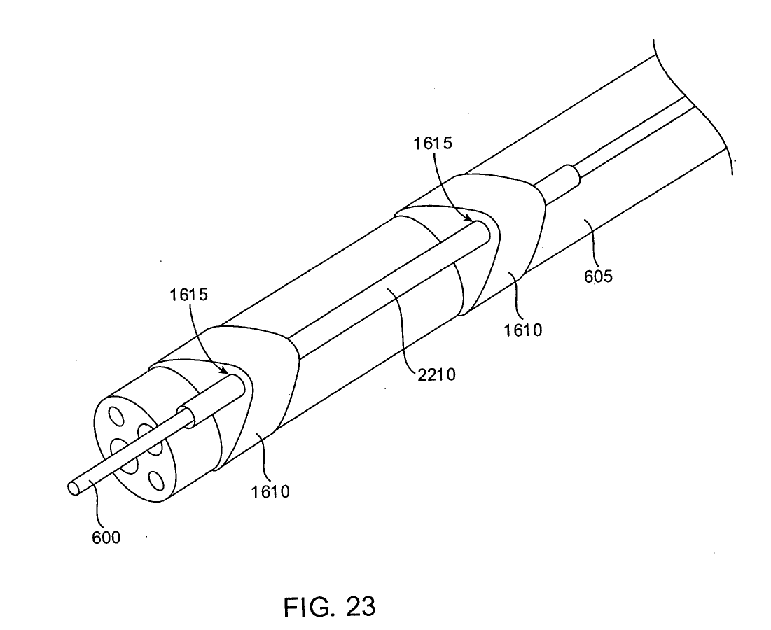

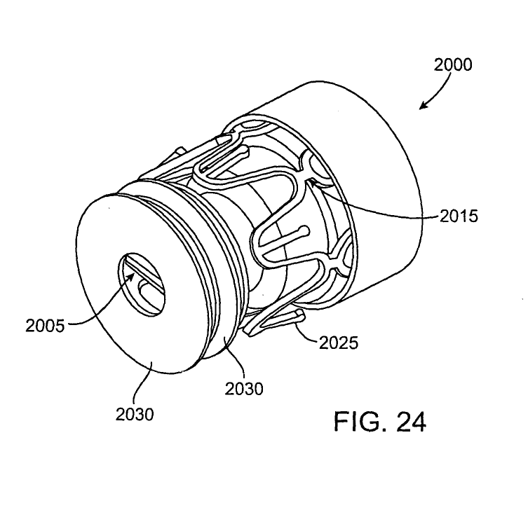

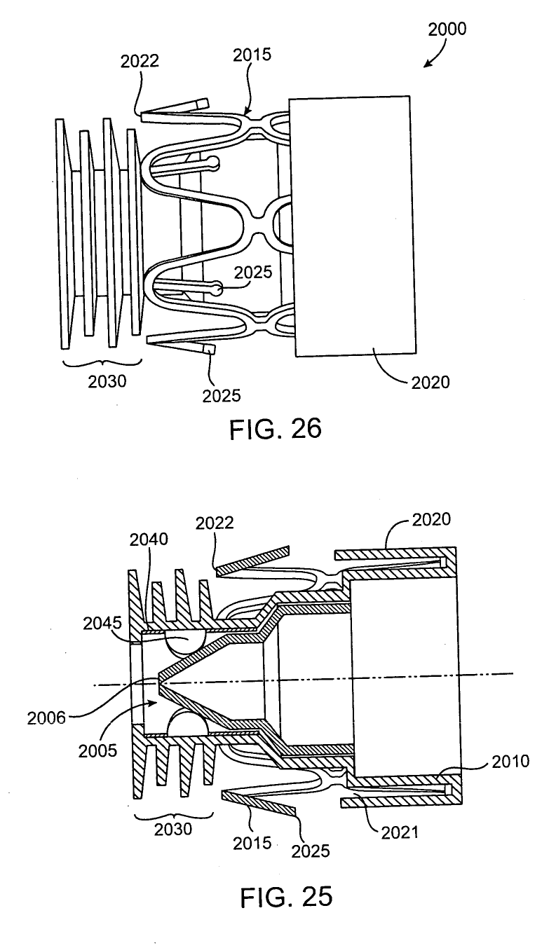

FIGS. 24-26 illustrates an exemplary one-way valve bronchial isolation device that can be mounted in a bronchial passageway for regulating fluid flow therethrough. FIG. 24 shows an isometric view of the device, FIG. 25 shows a cross sectional view of the device, and FIG. 26 is a side view of the device. The device has a flow control valve comprised of an elastomeric duckbill valve 2005, which can be manufactured of a material such as silicone. The duckbill valve includes a pair of movable, opposed walls or leaflets that meet at an apex position 2006 that can open and close. The walls can move to an open position in response to fluid flow in a first direction and move to a closed position in response to fluid flow in a second direction, which is generally opposite to the first direction. The valve 2005 is bonded inside a body 2010, which can also be manufactured of silicone. An adhesive can be used to bond the valve 2005 to the body 2010. The valve 2005 is a one-way valve, although two-way valves can also be used, depending on the type of flow regulation desired. The valve 2005 could also be replaced with an occluding member that completely blocks flow through the bronchial isolation device 2000.

A self-expanding retainer member 2015 is coupled to the body 2010. In one embodiment, the retainer member 2015 is manufactured from an elastic material, such as, for example, laser cut nickel titanium (Nitinol) tubing. The retainer member 2015 is comprised of a frame formed by a plurality of interconnected struts that define several cells. The retainer member 2015 can be expanded and heat treated to the diameter shown in order to maintain the super-elastic properties of the material. The retainer member 2015 is positioned inside a cuff 2020 of the body 2010 and retained therein by applying adhesive in the regions 2021 inside the distal cells of the retainer member.

The retainer member 2015 has proximal curved ends 2022 that are slightly flared. When the device is deployed inside a bronchial passageway, the proximal ends 2022 anchor with the bronchial wall and prevent migration of the device in the exhalation direction (i.e., distal-to-proximal direction). In addition, the retainer member 2015 has flared prongs 2025 that also anchor into the bronchial wall and serve to prevent the device from migrating in the inhalation direction (i.e., proximal-to-distal direction). Alternately, the retainer member 2015 can be manufactured of a material, such as Nitinol, and manufactured such that it changes shape at a transition temperature.

The bronchial isolation device 2000 includes a seal member that provides a seal with the internal walls of the bronchial passageway when the flow control device is implanted into the bronchial passageway. The seal member includes a series of radially-extending, circular flanges 2030 that surround the outer circumference of the bronchial isolation device 2000. When the device 2000 is implanted in a bronchial passageway, the seal member can seal against the bronchial walls to prevent flow past the device in either direction, but particularly in the inhalation direction. In one embodiment, the radial flanges are of different diameters in order to seal within passageways of different diameters.

With reference to FIG. 25, a rigid valve protector 2040 is bonded inside a proximal end of the body 2010 around the valve member 2005. The valve protector 2040 provides structural support to the radial flanges 2030 and protects the valve member 2005 from structural damage. In one embodiment, the valve protector 2040 is manufactured from nickel titanium tubing, although other rigid, biocompatible material would work, such as stainless steel, plastic resin, etc.

The valve protector 2040 can have two or more windows 2045 comprising holes that extend through the valve protector 2040, as shown in FIG. 25. The windows 2045 can provide a location where a removal device, such as rat-tooth graspers or forceps, can be inserted in order to facilitate removal of the bronchial isolation device 2000 from a bronchial passageway. In this regard, one jaw of the rat-tooth grasper is inserted into the valve protector 204, either above or below the valve, and the other jaw is pushed into the seal member. This allows the jaws of the grasper to close onto the wall of the valve protector 2040 and gain purchase through one of the windows 2045. The bronchial isolation device 2000 can be removed, for example, by pulling proximally on the device using the grasper.

It should be appreciated that the bronchial isolation device 2000 is merely an exemplary bronchial isolation device and that other types of bronchial isolation devices for regulating air flow can also be used. For example, the following references describe exemplary bronchial isolation devices: U.S. Pat. No. 5,594,766 entitled “Body Fluid Flow Control Device; U.S. patent application Ser. No. 09/797,910, entitled “Methods and Devices for Use in Performing Pulmonary Procedures”; and U.S. patent application Ser. No. 10/270,792, entitled “Bronchial Flow Control Devices and Methods of Use”. The foregoing references are all incorporated by reference in their entirety and are all assigned to Emphasys Medical, Inc., the assignee of the instant application.

Removal of Bronchial Isolation Devices from Bronchial Passageways

In some circumstances, it may be desirable to remove a previously-implanted bronchial isolation device. Disclosed are various devices and methods for removing a bronchial isolation device that has been implanted in a bronchial passageway. A first embodiment of a removable bronchial isolation device was described above with reference to FIGS. 24-26. As discussed, the bronchial isolation device 2000 includes grasping windows 2045. The grasping windows 2045 can be grasped using any of a variety of types of commercially-available, flexible grasping forceps. The forceps can be deployed into the bronchial passageway such as by deploying the forceps through the working channel of a flexible bronchoscope. In one embodiment, the forceps are a “rats-tooth” style that would provide a very firm purchase through the grasping windows 2045. Once the forceps grasp the flow control device through the grasping window 2045, the forceps are withdrawn to remove the device from the bronchial passageway.

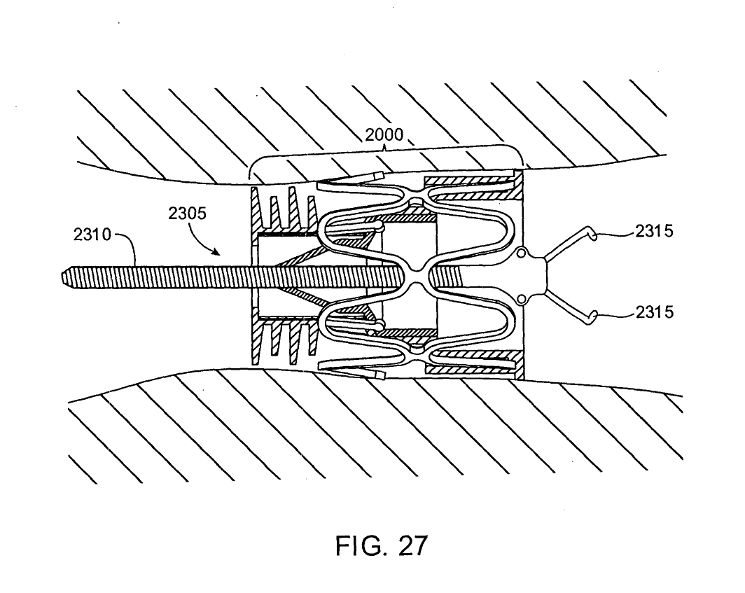

With reference to FIG. 27, there is now described a method of removing a bronchial isolation device 2000 that has been implanted in a bronchial passageway 2310. A removal device having an enlargeable engaging element on the distal region of the removal device is inserted through the bronchial isolation device such that the engaging element is positioned distally of the bronchial isolation device. The engaging element is then enlarged to a diameter that is larger than the diameter of at least a portion of the bronchial isolation device. The removal device is then moved in the proximal direction so that the enlarged engaging element inserts into, abuts, or otherwise engages the bronchial isolation device and pushes the bronchial isolation device in the proximal direction, thereby pushing and removing the bronchial isolation device out of the bronchial passageway.

In the embodiment shown in FIG. 27, the removal device comprises flexible forceps 2305 having an elongated delivery arm 2310 and jaws 2315 that may be opened relative to one another in a well-known manner. The forceps 2305 can be deployed to the bronchial passageway by advancing them through the working channel of a flexible bronchoscope. The jaws 2315 of the forceps 2305 are then opened to a diameter larger that that of the valve protector sleeve 2040. The forceps 2305 are withdrawn in the proximal direction while keeping the jaws 2315 open so that the jaws 2315 abut the implanted device. The open jaws 2315 push against the device and remove it. In another embodiment, the removal device comprises a delivery catheter and the enlargeable engaging element comprises an inflatable balloon on a distal region of the catheter. The bronchial isolation device can be removed by crossing the valve with the inflatable balloon, inflating the balloon to a diameter larger than the diameter of the bronchial isolation device 2000, and pulling the catheter proximally to remove the device.

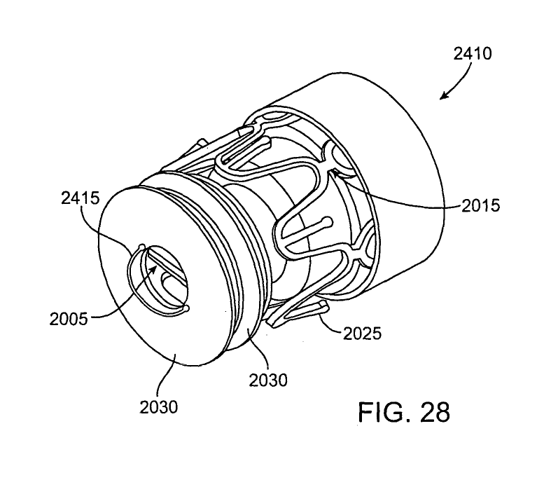

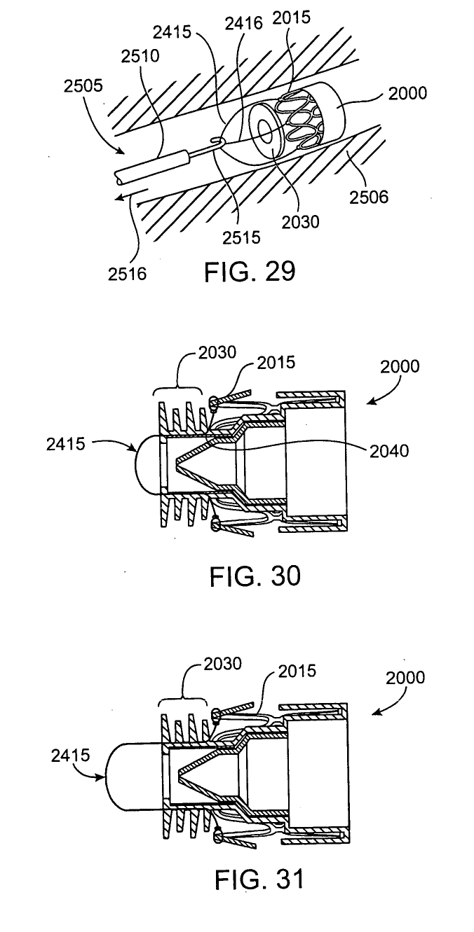

FIG. 28 shows an isometric view of a bronchial isolation device 2410 that includes a removal handle 2415 attached to a proximal side of the device 2410. The bronchial isolation device 2410 can have a similar or identical construction to the bronchial isolation device 2000 described above. The removal handle 2415 has a curved or straight contour that can be grasped by a removal device, such as forceps, for removing the bronchial isolation device 2410 from a bronchial passageway. The removal handle 2415 can be constructed of a flexible material, such as wire, suture, or of a rigid or semi-rigid material. The removal handle 2415 is attached at opposite ends to a portion of the bronchial isolation device, such as to the proximal radial flange 2030. In another embodiment, shown in FIG. 29, the removal handle 2415 is attached to the retainer member 2015, such as by threading the ends of the removal handle through the cells in the retainer member 2015.

As shown in FIG. 29, a removal device 2505 having an elongated, flexible shaft 2510, and a grasper 2515 is deployed to the location of the bronchial isolation device 2000 in the bronchial passageway 2506. The grasper 2515 has a structure that can couple to the removal handle 2415 for providing a force to the bronchial isolation device 2410 in at least the proximal direction 2516. In this regard, the grasper 2515 can be a hook, as shown in FIG. 29, or it can be movable jaws, forceps, or the like. Once the removal device 2505 is coupled to the removal handle 2415, the removal device 2505 is pulled in the proximal direction 2516 so that the graspers 2515 pull the bronchial isolation device 2410 out of the bronchial passageway. The force provided by the removal device 2505 can dislodge the bronchial isolation device 2410 from engagement with the bronchial walls. Alternately, the removal handle 2415 can be structurally coupled to predetermined points on the retainer member 2015 such that the retainer member radially collapses when a sufficient force is applied to the removal handle 2415 by the graspers 2515. Thus, the retainer will automatically disengage from the bronchial wall when a sufficient force is applied to the removal member.

It should be appreciated that in some circumstances pulling on the removal handle 2415 might apply an off-center load to the bronchial isolation device 2410, which may cause the device to tilt sideways in the bronchial passageway as the device is being removed. This may make removal more difficult. In a further embodiment, the removal device includes additional removal handles 2416 that are attached to the bronchial isolation devices in such a manner that the pulling force would be evenly distributed around the bronchial isolation device. The multiple removal handles 2415, 2416 may all be grasped by the graspers to reduce the likelihood of an off-center load. If the removal handles 2415, 2416 are grasped and pulled, the applied tension load is more balanced and allows the device to be pulled out more evenly. In addition, the graspers may be rotated or retracted to shorten the removal handles 2415, 2416 and cause the proximal end of the retainer member to be collapsed, thereby allowing the device to be pulled out easily.

FIG. 30 shows another embodiment of the bronchial isolation device 2410 wherein the removal handle 2415 is threaded between the valve protector 2040 and the radial flanges 2030. In this manner, no portion of the removal handle 2415 will be positioned between the radial flanges 2030 and the bronchial wall when the bronchial isolation device is deployed in a bronchial passageway eliminating any potential for leakage around the removal handle. In an alternate embodiment, shown in FIG. 31, the removal handle 2415 is threaded or sewn through the base of the radial flanges 2030 and bonded in place with an adhesive, such as silicone adhesive. The adhesive is adapted to break free when the removal handle is pulled, allowing the removal handle 2415 to pull on the retainer member.

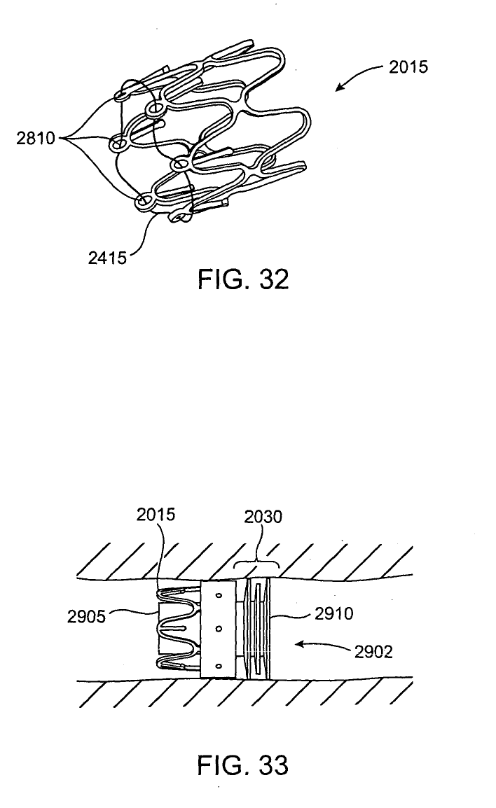

As discussed, the removal handle 2415 can be attached to the retainer member 2015 at one or more locations. FIG. 32 shows a retainer member 2015 that can be easily attached to a removal handle 2415. The retainer member 2015 includes at least one eyelet 2810 located on the proximal end of the retainer member 2015. A piece of elongated material can be threaded through the eyelets 2810 to form the removal handle 2415. In one embodiment, a plurality of eyelets are disposed around the periphery of the retainer member 2015. The removal handle loops through all of the eyelets 2810 so that the removal handle 2415 can be tensioned and constricted by applying a force thereto. When a sufficient proximal force is applied to the removal handle, the removal handle 2415 will constrict. The constriction will exert a radial force on the retainer and a sufficient force can be exerted to cause the retainer member 2015 to radially collapse.

In an alternative embodiment, the retainer member 2015 can be manufactured of a material, such as Nitinol, that changes shape at a transition temperature. In this regard, the retainer member 2015 can be configured to collapse when the retainer 2015 is exposed to a temperature that is below the material's transition temperature. After the bronchial isolation device is deployed, the temperature of the retainer 2015 can be reduced below the transition temperature, such as by introducing a chilled saline solution into the bronchial passageway where the device is deployed or by utilizing cryotherapy.

FIG. 33 shows a removable bronchial isolation device 2902 wherein the retainer member 2015 is disposed on a proximal side 2905 of the bronchial isolation device and the seal member (comprised of the radial flanges 2030) is disposed on a distal side 2910 of the bronchial isolation device. Thus, the proximal end of the retainer member 2015 is exposed in the proximal direction. The bronchial isolation device 2902 can be removed from the bronchial passageway by grasping the retainer member 2015 with graspers and pulling the bronchial isolation device.

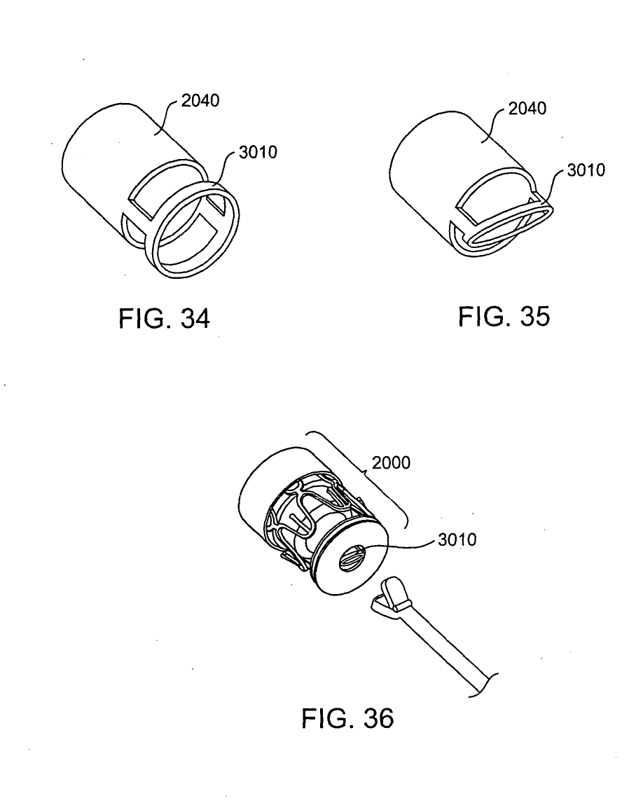

FIGS. 34-36 show a removal ring member 3010 that is attached to the proximal end of the valve protector 2040 (described above with reference to FIG. 25) of the bronchial isolation device. The valve protector 2040 is manufactured a temperature-sensitive material such as Nitinol. The valve protector 2040 has been heat treated so that it is in the shape configuration shown in FIG. 34 at a first temperature and in the shape configuration shown in FIG. 35 at a second temperature, which is different from the first temperature. In one embodiment, the first temperature is about the same as room temperature or about the same as body temperature (body temperature is 37 degrees Celsius). The second temperature is either greater than the first temperature of less than the first temperature, so that the valve protector 2040 is either heated or chilled to cause it to transition to the shape shown in FIG. 35. The first temperature and the second temperature can be varied according to desired shape-changing characteristics of the valve protector 2040.

Thus, at the first temperature, the removal ring member 3010 forms an open lumen through the valve protector to allow for passage of a guidewire and delivery system for delivery into the bronchial tree. Once the bronchial isolation device reaches the second temperature (by either cooling or heating the removal ring 3010), the removal ring 3010 deflects to a compressed state and forms into a shape that allows the removal ring 3010 to function as a removal handle, as shown in FIG. 35. A removal member, such as forceps, can then be used to grasp the compressed removal ring 3010 that is positioned in a bronchial isolation device 2000, as shown in FIG. 36.

Valve Devices for Use in Bronchial Wall Channels

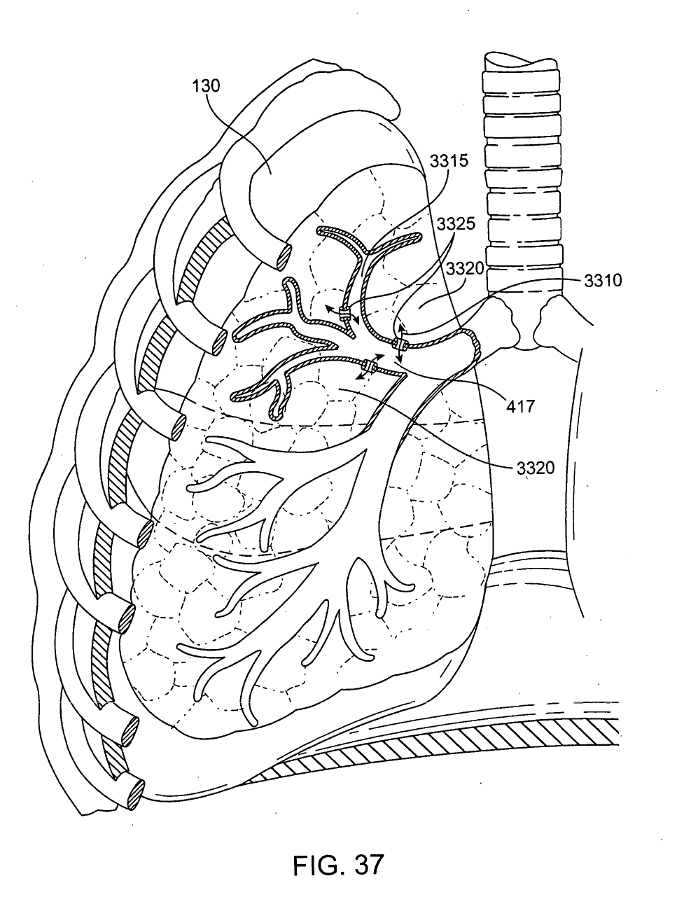

FIG. 37 shows a cutaway view of the human right lung. The right upper lobe 130 is shown with a number of channels 3310 cut through the wall of the bronchial passageway such that the channels 3310 fluidly connect the upper lobar bronchus 417 and segmental bronchus 3315 directly to the lung parenchyma 3320. Structural support devices 3325, such as grommets or stents, are positioned within the channels 3310 to keep the channels open and allow the flow of fluid in either direction through the channels 3310.

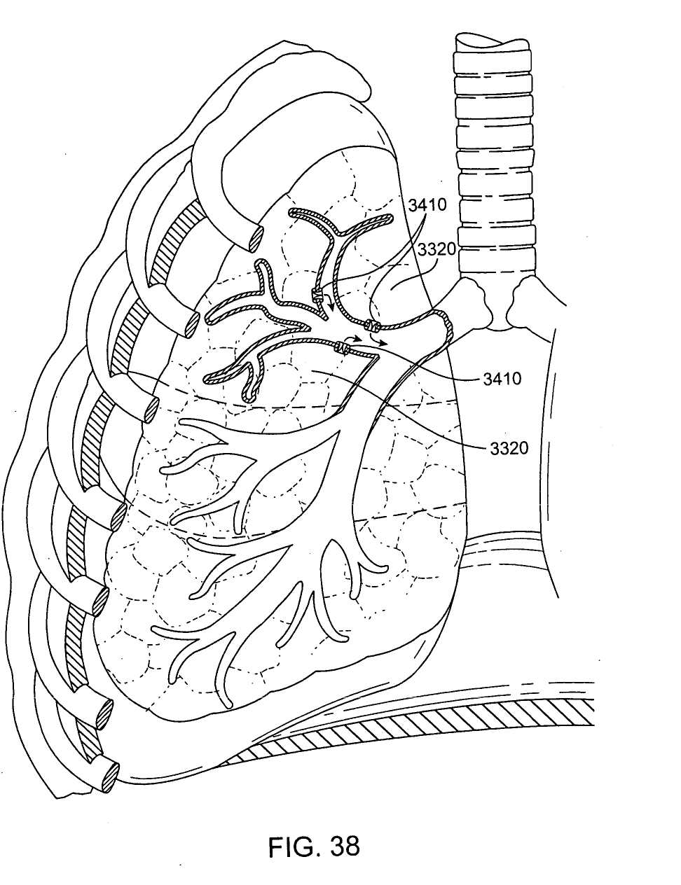

As discussed, it may be desirable to regulate the flow of fluid through the channels 3310. In this regard, a one-way or a two-way flow control device 3410 can be positioned within any of the channels, as shown in FIG. 38. The one-way flow control devices 3410 can be configured to restrict fluid flow in either direction through the channel. For example, the flow control device 3410 can be configured so that fluid can flow out of the distal lung tissue through the channels during exhalation, while preventing fluid from flowing back into the distal lung tissue through the channels during inhalation. This would reduce the likelihood of the diseased lung region becoming hyperinflated as a result of incoming fluid flow through the channels.

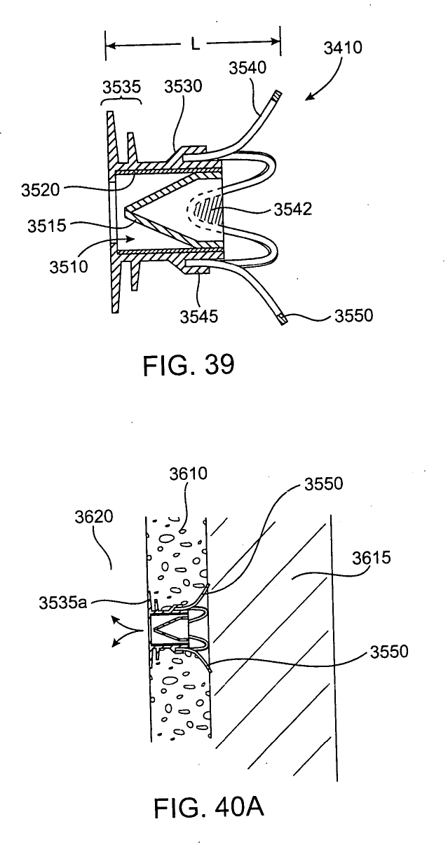

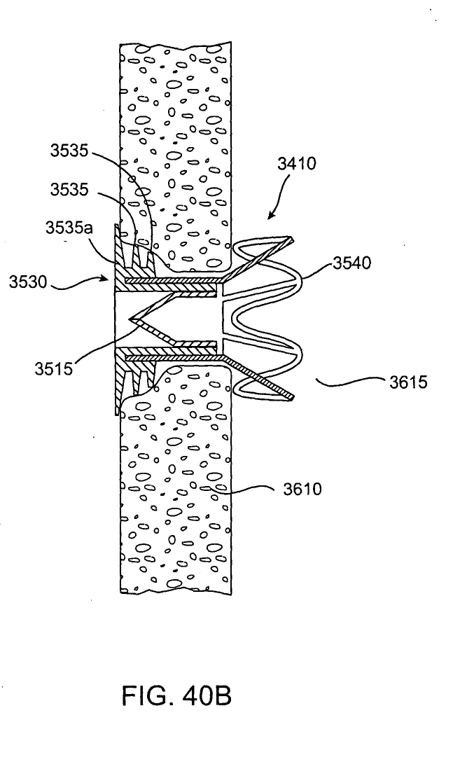

FIGS. 39 and 40A show a flow control device 3410 that is particularly suited for placement in a channel through a bronchial wall. FIG. 39 shows the flow control device 3410 in cross section and FIG. 40A shows the flow control device 3410 in cross-section mounted in a bronchial wall 3610 of a bronchial passageway. As shown in FIG. 40A, the flow control device 3410 anchors within the bronchial wall in a sealing fashion such that fluid in the bronchial passageway must pass through the flow control device 3410 in order to travel through the channel in the bronchial wall. The flow control device 3410 has fluid flow regulation characteristics that can be varied based upon the design of the flow control device. For example, the flow control device 110 can be configured to either permit fluid flow in two directions, permit fluid flow in only one direction, completely restrict fluid flow in any direction through the flow control device, or any combination of the above. For example, as shown in FIG. 40A, the flow control device 3410 allows fluid to flow from the parenchyma 3615 into the lumen 3620 of the bronchial passageway. The flow control device 3410 can be configured such that when fluid flow is permitted, it is only permitted above a certain pressure, referred to as the cracking pressure.

With reference to FIG. 39, the flow control device 3410 includes a tubular main body, such as an annular valve protector 3520 that defines an interior lumen 3510 through which fluid can flow. The flow of fluid through the interior lumen 3510 is controlled by a valve member 3515, such as a duckbill valve, which is mounted inside the annular valve protector 3520. The valve member 3515 is configured to allow fluid flow through the interior lumen 3510 in a first direction and restrict fluid flow through the interior lumen 3510 in a second direction. The valve member 3515 can be a separate piece from the tubular body or it can be integrally formed with the tubular body. An elastomeric seal member 3530 includes one or more radial flanges 3535 that can form a seal with the bronchial wall 3610. The radial flanges 3535 are located on a first end of the flow control device 3410, although it should be appreciated that the flanges 3535 can be located on the second end or on other locations along the length of the flow control device 3410. The flanges 3535 can be separate pieces from the tubular body or they can be integrally formed with the tubular body

As shown in FIG. 40A, the flow control device 3410 is positioned within a channel in the bronchial wall 3610 such that the interior lumen 3510 communicates with the bronchial passageway. The flange 3535a is positioned and shaped so that it engages the interior surface of the bronchial wall 3610. The flange 3535a forms a seal with an inner surface of the bronchial wall so that fluid must flow through the valve 3515 in order to flow through the channel in the bronchial passageway. Thus, the seal member 3530 prevents fluid in the bronchial passageway from leaking through the channel in the bronchial wall 3610.

The flow control device 3410 also includes a retainer member 3540, such as a stent, that is coupled to the tubular main body and that functions to anchor the flow control device 3410 within the channel in the bronchial wall. The retainer member 3540 is positioned within an annular flap 3545 and secured therein, such as by using adhesive 3542 located within the flap 3545. In an alternative design, the valve protector 3520 and the retainer member 3540 could be laser cut from a single piece of material, such as Nitinol tubing, and integrally joined, thereby eliminating the adhesive joint.

The retainer member 3540 has a structure that can contract and expand in size (in a radial direction and/or in a longitudinal direction) so that the retainer member 3540 can expand to grip the bronchial wall 3610 in which it is mounted. In this regard, the retainer member 3540 can be formed of a material that is resiliently self-expanding. In the embodiment shown in FIGS. 39 and 40A, the retainer member 3540 comprises a contoured frame that surrounds the flow control device 3410. The frame has one or more loops or prongs 3550 that can expand outward to grip the bronchial wall 3610 and compress at least a portion of the seal member 3550 against the bronchial wall 3610. As shown in FIG. 40A, the prongs 3550 are sized and shaped so that they form a flange that engages the exterior surface of the bronchial wall 3610 (the surface that is adjacent the parenchyma 3615). The prongs 3550 and the flange 3535a form flanges that collectively grip the bronchial wall 3610 therebetween to secure the flow control device 3410 to the bronchial wall 3610.

The flow control device 3410 has dimensions that are particularly suited for sealing, retention and removability in a bronchial wall channel placement. As shown in FIG. 40A, the flow control device 3410 has a general outer shape and dimension that permit the flow control device to fit entirely within the channel in the bronchial wall 3610. In this regard, the flow control device 3410 has a length L that is suited for placement within a channel in the bronchial wall. In one embodiment, the length L is substantially the same as the thickness of the bronchial wall 3610. Alternately, the length L can be slightly less or slightly greater than the thickness of the bronchial wall 3610. The thickness of the bronchial wall 3610 can vary based on the patient and on the bronchial passageway's location in the bronchial tree. In general, the bronchial wall thickness is in the range of about 0.1 millimeters to about 5 millimeters. In one embodiment, the bronchial wall thickness is about 1 millimeter.

The valve member 3515 can be made of a biocompatible material, such as a biocompatible polymer including silicone. The seal member 3530 is manufactured of a deformable material, such as silicone or a deformable elastomer. The retainer member 3540 is desirably manufactured of an elastic material, such as Nitinol.

FIG. 40B shows another embodiment of the flow control device 3410 wherein a portion of the retainer member 3540 is annular and concentrically surrounds an annular portion of the seal member 3530. The valve member 3515 is positioned within the seal member 3530. The flanges 3535 forms successive seals with the bronchial wall 3610, with a front-most flange 3535a engaging the interior surface of the bronchial wall 3610. The retainer member 3540 extends into the parenchyma 3615 and engages the surface of the bronchial wall 3610 that is adjacent the parenchyma 3615.

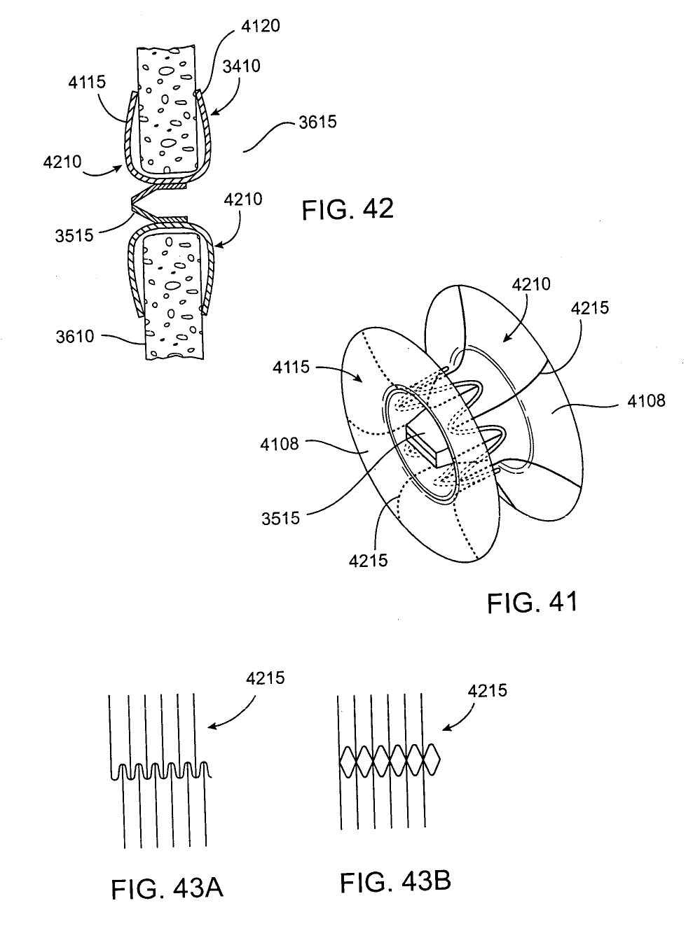

FIGS. 41 and 42 show a further embodiment of the flow control device 3410. FIG. 41 shows a perspective view of the flow control device 3410 and FIG. 42 shows a cross-sectional view of the flow control device 3410 mounted in a channel of a bronchial wall 3610. The flow control device 3410 includes a one-way valve member 3515, such as a duckbill valve. The valve member 3410 is mounted within an anchor and seal member 4210 comprised of a tubular body that can both anchor the flow control device 3410 to the bronchial wall 3610 and form a seal with the bronchial wall 3610.

The anchor and seal member 4210 includes a retainer 4215 that includes a plurality of prongs (shown in FIG. 42) that are shaped so as to form a front flange 4115 and a rear flange 4120. The front flange 4115 and the rear flange 4120 engage the inner and outer surfaces of the bronchial wall 3610 to secure the flow control device 3410 in a fixed position. In addition, the flanges 4115, 4120 form a seal with the bronchial wall 3610 so that fluid cannot flow between the flanges and the bronchial wall 3610, but must rather flow through the valve member 3515 in order to flow through the channel in the bronchial wall 3610.

With reference to FIG. 41, the prongs of the retainer 4215 are spaced from one another to form a plurality of cells that are filled with a membrane 4108 that is made of a material that can form a seal with the bronchial wall 3610. The membrane 4108 can be made of silicone, polyurethane, or some other material that can form a seal. The membrane 4108 can be formed by dipping the entire retainer 4215 into a bath of the membrane material so that the material dries within the cells and forms the membrane. FIGS. 43A and 43B show the retainer 4215 in a planar view, wherein the prongs are laser cut from a tube of material and if the tube were unrolled after laser cutting and heat-treated into the shape shown in FIGS. 43A and 43B. The retainer 4215 can then be formed into the shape shown in FIG. 41.

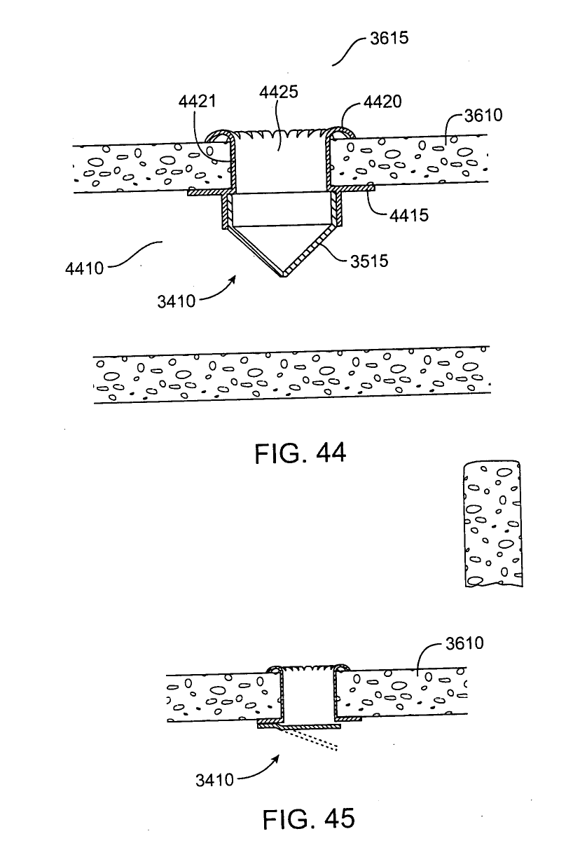

FIG. 44 shows a further embodiment of the flow control device 3410, shown mounted in a bronchial wall 3610 of a bronchial passageway. In this embodiment, the valve member 3515 is located entirely within the lumen 4410 of the bronchial passageway. The flow control device 3410 includes an inner flange 4415 that engages the interior surface of the bronchial wall 3610 and an outer flange 4420 that engages the exterior surface of the bronchial wall 3610. The outer flange 4420 can be formed by crimping a deformable material against the bronchial wall, such as by using an expandable balloon or other device located in the parenchyma 3615. Alternatively, the outer flange 4420 may be manufactured from a resilient material such as Nitinol such that the flow control device 3410 could be constrained within a delivery device for insertion through the channel in the bronchial wall 3610, and then released so that the outer flange 4420 springs into contact with the outside of the bronchial wall and assumes the shape shown in FIG. 44

A tubular body 4421 has first and second ends on which the inner flange 4415 and outer flange 4420, respectively, are positioned. The tubular body 4421 has a passage or flow channel 4425 therethrough and is configured to extend through the bronchial wall 3610 with the channel 4425 in communication with the lumen 4410 of the bronchial passageway 3610.

As mentioned, the valve member 3515 in FIG. 44 is located entirely within the bronchial lumen 4410. This allows the flow channel 4425 of the flow control device 3410 to be maximized for a given diameter of the channel in the bronchial wall 3610 in that the valve member 3515 does not consume any of the volume of the flow channel 4425. FIG. 44 shows the valve member 3515 as a duckbill valve. Other types of valves can also be used with the flow control device 3410. For example, FIG. 45 shows the flow control device 3410 with the valve member 3515 comprising a flap valve having a hinged flap or leaflet that opens in response to fluid flow in a first direction and closes in response to fluid flow in a second direction.

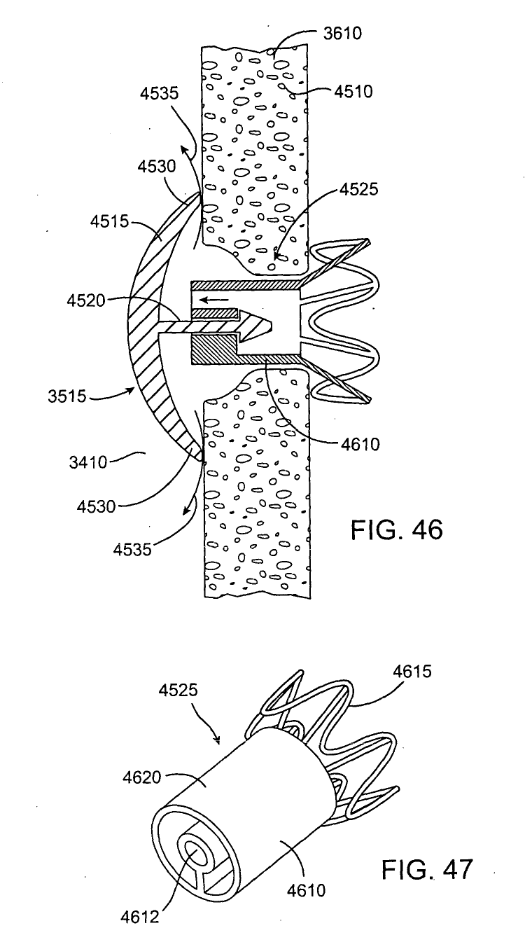

FIG. 46 shows another embodiment of the flow control device 3410 where the valve member 3515 is positioned within the bronchial lumen. The valve member 3515 in the embodiment of FIG. 46 is comprised of an umbrella valve that includes an dome-shaped umbrella 4515 having an elongate mounting member 4520 that is coupled to a seal and retainer member 4525. The umbrella 4515 has peripheral edges 4530 that sealingly engage the interior surface of the bronchial wall 4510. The edges 4530 are biased toward the bronchial wall 4510 so that they press against the bronchial wall 4510 in a default state and prevent fluid from flowing through the channel in the bronchial wall 4510. When exposed to a sufficient fluid pressure, the edges 4530 lift away from the bronchial wall 3610 and permit fluid to flow between the bronchial wall and the edges 4530, as exhibited by the arrows 4535 in FIG. 46.

FIG. 47 shows a perspective view of the seal and retainer member 4525 of the flow control device 3410 shown in FIG. 46. The seal and retainer member 4525 has a cylindrical portion 4610 that sealingly engages the bronchial wall 3610 to prevent flow between the cylindrical portion 4610 and the bronchial wall 3610. A retainer portion comprised of prongs 4615 is located at the end of the cylindrical portion 4610. The retainer portion engages the bronchial wall 3610 to secure the flow control device in the channel of the bronchial wall 3610. The cylindrical portion 4610 includes a coupling passage 4612 into which the mounting member 4520 of the umbrella 4515 can be coupled. The cylindrical portion 4610 also defines an interior lumen 4620 through which fluid can flow.

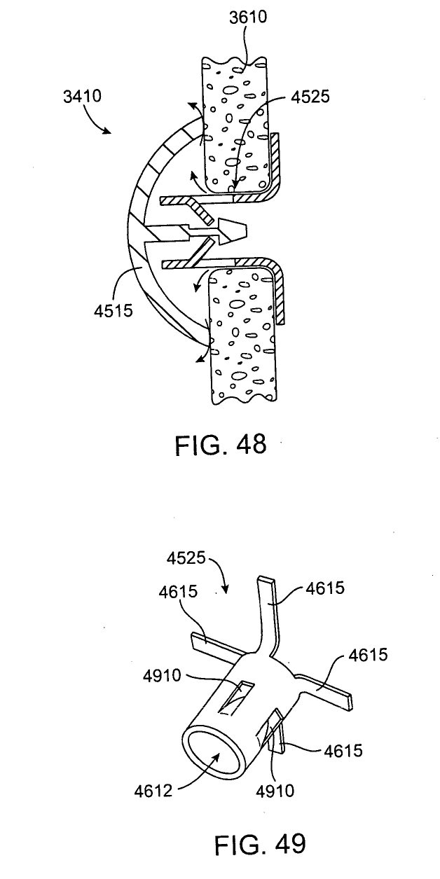

FIG. 48 shows another embodiment of the flow control device 3410 mounted in a bronchial wall 3610. This embodiment is similar to the embodiment shown in FIG. 46 in that it includes an umbrella valve having an umbrella 4515. The umbrella 4515 is coupled to a retainer and seal member 4525 that is positioned within a channel in the bronchial wall 3610. As shown in FIG. 49, the retainer and seal member 4525 includes a cylindrical portion 4610 that defines an internal lumen 4612 for the passage of fluid. One or more fluid entry ports 4910 comprised of holes are disposed on the cylindrical portion to allow fluid to flow into the internal lumen 4612. The retainer and seal member 4525 includes a plurality of prongs 4615 that have a flattened configuration that can engage the surface of the bronchial wall 3610, as shown in FIG. 48.

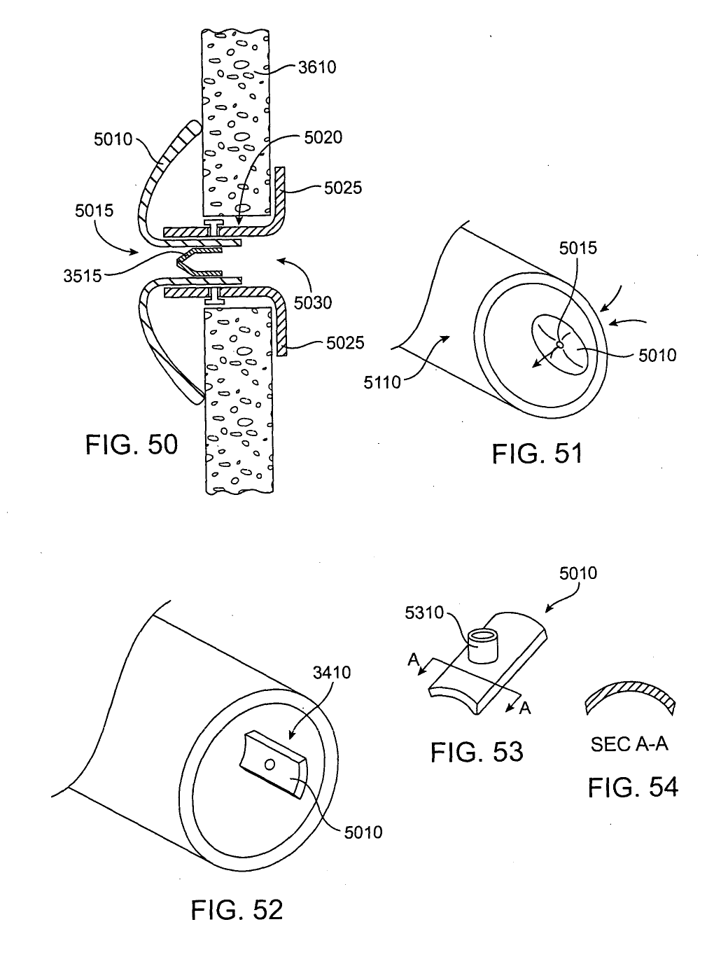

FIG. 50 shows yet another embodiment of the flow control device 3410 mounted in a bronchial wall 3610. This embodiment combines a dome-shaped diaphragm 5010 with a valve member 3515 comprised of a duckbill valve. The diaphragm 5010 includes a centrally-located, internal tunnel 5015, in which is mounted the valve member 3515. The diaphragm 5010 is coupled to a retainer member 5020 that has a tubular portion positioned in the channel of the bronchial wall 3610 and a flange 5025 that engages the bronchial wall 3610. The diaphragm 5010 and the flange 5025 hold the bronchial wall 3610 therebetween to secure the device within the channel in the bronchial wall 3610. The flange 5025 can be formed by crimping a deformable material against the bronchial wall, such as by using an expandable balloon or other device located in the parenchyma 3615 in a manner similar to that shown in FIGS. 44 and 45. Alternatively, the flange 5025 may be manufactured from a resilient material such as Nitinol such that the flow control device 3410 could be constrained within a delivery device for insertion through the channel in the bronchial wall 3610, and then released so that the flange 5025 springs into contact with the outside of the bronchial wall and assumes the shape shown in FIG. 50.

The valve member 3515 regulates fluid flow through an internal lumen 5030 that is collectively formed by the tunnel 5010 of the diaphragm 5010 and the cylindrical portion of the retainer member 5020.

FIG. 51 shows a cutaway, perspective view looking into a bronchial passageway 5110 in which the flow control device 3410 of FIG. 50 is mounted. The diaphragm 5010 is positioned within a lumen of the bronchial passageway 5110 such that the diaphragm 5010 seals with and is engaged against the inner surface of the bronchial wall. The tunnel 5015 provides a passageway for fluid to flow into the lumen of the bronchial passageway 5110.

FIG. 52 shows another embodiment of the flow control device 3410 of FIG. 50, wherein the diaphragm 5010 has a rectangular shape. As shown in FIG. 53, the rectangular diaphragm 5010 has a mounting member 5310 that can be coupled to the retainer member 5020. FIG. 54 shows a cross-sectional view of the diaphragm 5010 and illustrates that the diaphragm has a curved contour which conforms to the curved contour of the bronchial wall in which the flow control device is mounted in order to assist the sealing between the diaphragm 5010 and the bronchial wall.

The channels in the bronchial wall can be created in a variety of different manners. In one embodiment, a cutting catheter with a sharpened tip, such as up to 5 mm in diameter, can be used to puncture the bronchial wall. In another embodiment, a stiff guidewire delivered via the inner lumen of a flexible bronchoscope can be used to puncture the bronchial wall. In another embodiment, a flexible biopsy forceps is placed through the working channel of the bronchoscope and used to cut a hole through the bronchial wall. In yet another embodiment, RF energy is delivered to the bronchial wall at the distal end of a catheter and used to create a hole in the bronchial wall. The RF method would also cauterize the hole in the bronchial wall, thus stopping blood flow and sealing the channel.

Once the channel is formed in the bronchial wall, the flow control device 3410 is delivered into the channel. In one embodiment, a guidewire is first placed through the channel, and then a delivery catheter containing the flow control device 3410 is advanced over the guidewire, and into the channel. The guidewire can be placed using the working channel of a flexible bronchoscope, can be guided freehand, or can be placed by any other suitable method. In another embodiment, a guiding catheter is inserted into the channel, and the flow control device 3410 is pushed through the catheter and into position in the channel. If the flow control device 3410 can be compressed into the tip of a delivery catheter, the delivery catheter can be advanced through the working channel of a flexible bronchoscope, inserted into the channel and the device 3410 deployed. In yet another embodiment, the flow control device 3410 is grasped with forceps or some other tool and inserted into the channel.

Once the flow control device 3410 is delivered to the location of the channel, a first end of the flow control device is inserted through the channel in the wall so that the interior lumen 3510 is in communication with the bronchial passageway. The flow control device is then secured in the wall so that it allows fluid flow in a first direction through the passage to or from the bronchial passageway. The flow control device is configured to restrict fluid flow in a second direction through the passage to or from the bronchial passageway. The flow control device 3410 provides a seal between the flow control device 3410 and the bronchial wall to restrict fluid flow therebetween. The seal can be provided by a flange on the flow control device that engages an inner or outer surface of the bronchial wall. A stent of the flow control device can be expanded to engage the bronchial wall.

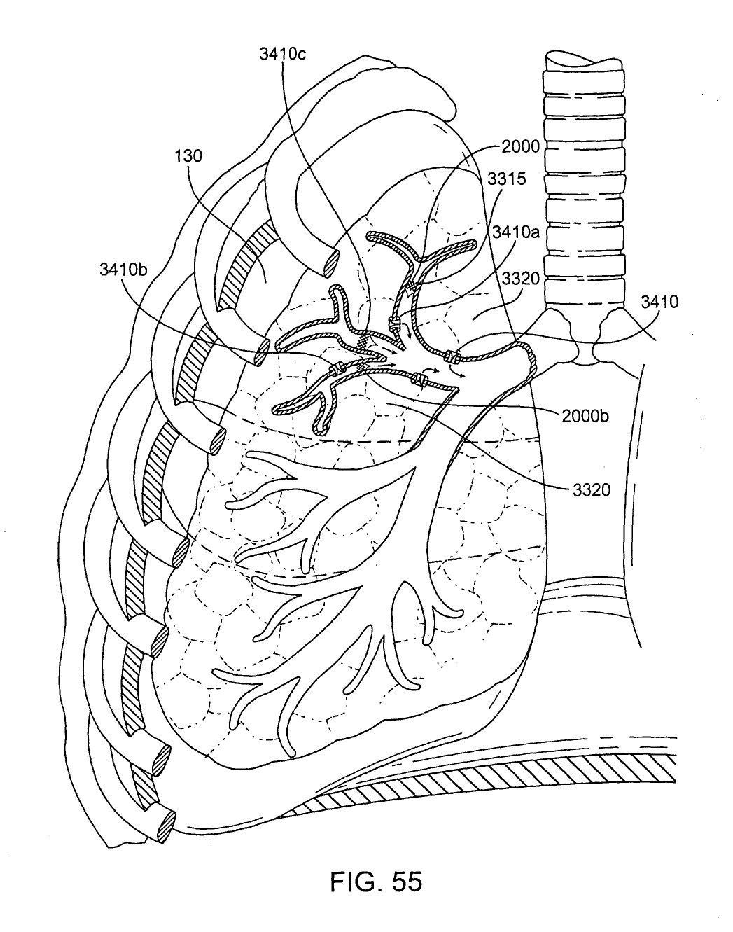

One or more of the flow control devices 3410 can be used in combination with the bronchial isolation devices 2000 described above with reference to FIGS. 20-22 in order to modify the fluid flow dynamic to a lung region. For example, FIG. 55 shows flow control devices 3410 implanted in various bronchial walls in the right upper lobe 130. In addition, bronchial isolation devices 2000 are mounted within various bronchial passageways. The flow control devices 3410 permit flow in a first direction and restrict flow in a second direction through the bronchial wall channels in which the devices 3410 are mounted. For example, the flow control devices 3410 can permit flow from the parenchyma 3320 into the bronchial passageway, but prevent flow in the opposite direction.

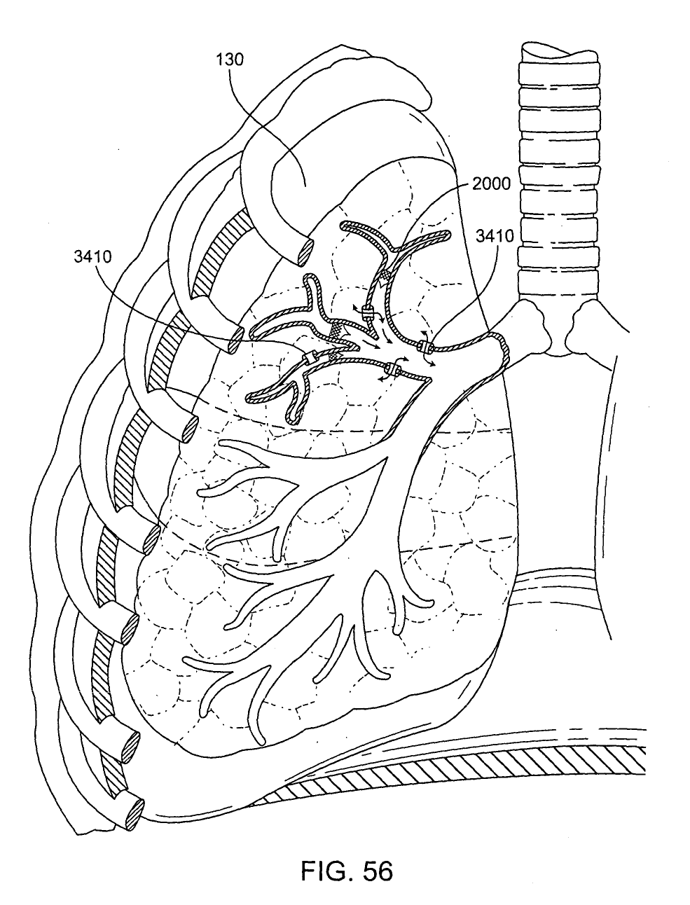

A desired fluid flow dynamic to a lung region can be achieved by deploying various combinations of flow control devices 3410 and bronchial isolation devices 2000 in one or more bronchial passageways that communicate with the lung region. A flow control devices 3410 can be mounted in the same bronchial passageway in which a bronchial isolation device 2000 is mounted, or a flow control device 3410 can be mounted in a different bronchial passageway, or a combination thereof can be used. For example, FIG. 55 shows a bronchial isolation device 2000a and a flow control device 3410a both mounted in the same segmental bronchus 3315. A flow control device 3410c is mounted in a different segmental bronchus. The positioning of the flow control device 3410 relative to the bronchial isolation device 2000 in the bronchial tree can also vary. A channel in which the flow control device 3410 is deployed can be positioned either proximally or distally of a bronchial isolation device 2000 in a bronchial passageway. For example, the flow control device 3410a is in a channel that is located proximally of the bronchial isolation device 2000a, and the flow control device 3410b is in a channel that is located distally of the bronchial isolation device 2000b. FIG. 56 shows flow control devices 3410 implanted in various bronchial walls in the right upper lobe 13 wherein the devices 3410 permit flow in both directions. A combination of two-way flow control devices 3410 and one-way flow control devices 3410 can be used to achieve a desired fluid flow dynamic to and from a lung region.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed:

1. A method of removing a flow control device implanted in a bronchial passageway of a patient, comprising:
providing a removal device having an elongate shaft and a distal engaging element;
inserting the removal device through the bronchial isolation device such that the distal engaging element is positioned within or distally of the bronchial isolation device in the bronchial passageway;
transitioning the engaging element of the removal device to have a radial size that is larger than the radial size of at least a portion of the bronchial isolation device;
engaging the bronchial isolation device with the distal engaging element to urge the bronchial isolation device in the proximal direction, thereby removing the bronchial isolation device out of the bronchial passageway.

2. A method as defined in claim 1, wherein the engaging element of the removal device comprises a set of jaws that can be opened to enlarge the distal region of the removal device.

3. A method as defined in claim 1, wherein the enlargeable distal region of the removal device comprises a balloon that can be inflated to enlarge the distal region of the removal device.

4. A removable flow control device for implanting in a bronchial passageway, comprising:
a valve member that regulates fluid flow through the flow control device;
a seal member that at least partially surrounds the valve member, wherein the seal member seals with the interior wall of the bronchial passageway when the flow control device is implanted in the bronchial passageway;
a retainer member secured to the seal member, wherein the retainer member exerts a radial force against the interior wall of the bronchial passageway when the flow control device is implanted in the bronchial passageway and retains the flow control device in a fixed location in the bronchial passageway; and
a first removal handle attached to the retainer member, the handle being configured to collapse the retainer member upon application of a force to the handle member.

5. A device as defined in claim 4, wherein the retainer member comprises a frame having at least one eyelet and wherein the removal handle is threaded through the eyelet.

6. A device as defined in claim 5, wherein a plurality of eyelets are positioned around a periphery of the retainer member, the handle being threaded through the plurality of eyelets.

7. A device as defined in claim 4, wherein the removal handle can be tensioned to apply a constriction force to the retainer member for radially collapsing the retainer member.

8. A device as defined in claim 1, wherein the removal handle comprises a flexible suture.

9. A device as defined in claim 1, wherein the removal handle is attached to at least two locations on the retainer member so that the removal handle can apply a distributed force to the retainer member.

10. A device as defined in claim 1, wherein the at least two locations are located on a periphery of the retainer member.

11. A device as defined in claim 1, wherein the removal handle is a shape memory material.

12. A device as defined in claim 11, wherein the removal handle has a first shape at room temperature and a second shape at body temperature.

13. A device as defined in claim 11, wherein the removal handle has a first shape at body temperature and a second shape at a second temperature other than body temperature.

14. A device as defined in claim 13, wherein the second temperature is cooler than body temperature.

15. A device as defined in claim 13, wherein the second temperature is warmer than body temperature.
